(19) United States
(12) Patent Application Publication
(10) Pub. No.: US 2026/0199635 A1
(43) Pub. Date: Jul. 16, 2026

(54) CATHETER STABILIZATION DEVICES, SYSTEMS, AND METHODS

(71) Applicant: VASONICS, INC., Corona, CA (US)

(72) Inventor: Ramy Albany, Corona, CA (US)

(21) Appl. No.: 19/135,315

(22) PCT Filed: Dec. 14, 2023
(86) PCT No.: PCT/US2023/084114

§ 371 Date: Jun. 3, 2025

Related U.S. Application Data
(60) Provisional application No. 63/387,670, filed on Dec. 15, 2022.
(60) Provisional application No. 63/600,566, filed on Nov. 17, 2023.

(51) Int. Cl. A61M 25/02; A61M 25/00

(52) U.S. Cl. CPC A61M 25/02; A61M 25/0097; A61M 2025/024; A61M 2025/0246; A61M 2025/0266; A61M 2025/028

(57) ABSTRACT

A device configured for use at a catheter insertion site on a subject can include a base configured to be secured to the subject's skin over the insertion site and a catheter hub holder configured to secure to a catheter hub connected to a catheter that is configured for insertion at the insertion site. The catheter hub holder can be movably coupled to the base to allow for relative movement between the base and the catheter hub and catheter. The catheter hub holder and base can be connected via a gimbal arrangement. The base can include a membrane configured to secure to the subject's skin and a frame. In some implementations, the catheter hub holder is rotatably coupled to a clamp that is connected to the frame. In some implementations, the clamp is pivotably connected to the frame and configured to pivot between open and closed positions.

### INCORPORATION BY REFERENCE TO ANY PRIORITY APPLICATIONS

This application claims the benefit of U.S. Provisional Application No. 63/600,566, filed Nov. 17, 2023, and U.S. Provisional Application No. 63/387,670, filed Dec. 15, 2022. Each of the foregoing applications is hereby incorporated by reference in its entirety.

### TECHNICAL FIELD

In general, the present disclosure relates to catheter stabilization devices, systems, and methods.

### BACKGROUND

Intravenous catheter procedures have been utilized on patients for over a century. Peripheral Intravenous Catheters (PIVC) and Peripherally Inserted Central Catheters (PICC) are commonly employed on patients within a hospital or other care facility to provide medication, nutrition, or fluids. After cleaning a catheter insertion site, a needle is inserted into a vein of a patient and a catheter is then inserted into the vein. Such catheter is typically connected to a catheter hub.

### SUMMARY OF CERTAIN ASPECTS

Stabilizing a catheter at a catheter insertion site is important to inhibit, among other things, catheter dislodgement, and to ensure that the purpose for which the catheter was employed is achieved (for example, intravenous therapy). Traditional techniques of stabilizing a catheter involve applying one or more adhesive film dressings directly over a catheter hub connected to the catheter and the catheter insertion site. Such techniques have a variety of drawbacks. For example, such techniques do not sufficiently inhibit catheters from becoming dislodged (for example, due to patient movement) and from being pushed further into a patient's arm (or other body part). The adhesive dressings utilized in such techniques cover and incubate germs and bacteria at the insertion site, which can lead to various infections and complications. Frequent replacement of such adhesive dressings or the catheter can increase risk of bacterial infection and cause irritation and trauma to the catheter insertion site (especially with geriatric patients). Traditional techniques also inhibit normal skin respiration and ventilation, raise humidity and temperature at the insertion site (which can promote microorganism growth), cause the tip of the catheter to be improperly angled within the vein, obscure the catheter insertion site (thereby inhibiting assessment of the status of the catheter and insertion site), and often cause “kinking” of the catheter.

Various implementations of devices, systems, and methods for stabilizing a catheter at a catheter insertion site are disclosed herein which address one or more of the above-described shortcomings, among others. Various implementations of the devices disclosed herein are configured to stabilize a catheter at a catheter insertion site and inhibit relative movement between the catheter and the vein into which the catheter extends and/or the catheter insertion site. This in turn improves the integrity and condition of the catheter insertion site and the vein into which the catheter is inserted. For example, various implementations of the devices disclosed herein include a first portion configured to be secured to a subject's skin and a second portion that is movably coupled to the first portion and that is configured to be secured to a catheter hub connected to a catheter. Allowing relative movement between such first and second portions of the device allows for relative movement between the first portion (which secures to the subject's skin) and the catheter hub and connected catheter. The vein into which the catheter is inserted may not move proportionally with the skin to which such first portion is secured. Accordingly, allowing for relative movement between the first portion of the device and the catheter hub and connected catheter can inhibit relative movement between the catheter and the vein. Such arrangement can inhibit the catheter hub and connected catheter from being moved and making contact with the vein wall if the first portion of the device is moved (for example, by an inadvertent force).

Disclosed herein is a catheter stabilization device configured for use at a catheter insertion site on skin of a subject, the catheter stabilization device comprising: a first portion configured to secure to one or more portions of the subject's skin; and a second portion configured to secure to a catheter hub coupled to a catheter that is configured for insertion at said catheter insertion site; wherein said first and second portions of the catheter stabilization device are movably coupled to one another to allow for relative movement between said first portion of the catheter stabilization device and said catheter hub when said catheter stabilization device is in use and said catheter is inserted at said catheter insertion site.

In some implementations, said first portion is configured to surround the catheter insertion site when secured to said one or more portions of the subject's skin. In some implementations, said second portion does not contact the subject's skin when said first portion is secured to said one or more portions of the subject's skin. In some implementations, at least a portion of said first portion of the catheter stabilization device configured to contact said one or more portions of the subject's skin is made of a first material, and wherein said second portion of the catheter stabilization device is made of a second material that is more rigid than said first material. In some implementations, said second portion is configured to move relative to said first portion along a first axis and a second axis that is perpendicular to said first axis. In some implementations, said second portion is configured to rotate about at least one of said first axis and said second axis. In some implementations, said second portion is configured to rotate about said at least one of said first axis and said second axis an amount that is between about 5 degrees and about 45 degrees. In some implementations, said second portion is configured to rotate about said at least one of said first axis and said second axis an amount that is between about 5 degrees and about 30 degrees. In some implementations, said second portion is configured to rotate about each of said first axis and said second axis. In some implementations, said second portion is further configured to move relative to said first portion along a third axis that is perpendicular to both of said first axis and said second axis. In some implementations, said second portion is configured to rotate about each of at least two of said first axis, said second axis, and said third axis. In some implementations, said second portion is configured to rotate about each of at least two of said first axis, said second axis, and said third axis an amount that is between about 5 degrees and about 45 degrees. In some implementations, said second portion is configured to rotate about each of at least two of said first axis, said second axis, and said third axis an amount that is between about 5 degrees and about 30 degrees.

In some implementations, said second portion is configured to move relative to said first portion along a first axis and a second axis that is perpendicular to said first axis, and wherein movement of said second portion relative to said first portion along a third axis that is perpendicular to both of said first and second axes is inhibited. In some implementations, said second portion is configured to move relative to said first portion along a first axis that is perpendicular to both of a second axis and a third axis, and wherein movement of said second portion relative to said first portion along said second and third axes is inhibited. In some implementations, the second portion of the catheter stabilization device is configured to secure to said catheter hub such that relative movement between said second portion and said catheter hub is inhibited.

In some implementations: said first portion comprises at least one receptacle having an interior; and said second portion comprises a body and at least one arm extending outward from said body, said body configured to secure to said catheter hub, at least a portion of said at least one arm configured to be positioned in said interior of said at least one receptacle; and said first and second portions of the catheter stabilization device are movably coupled to one another via said at least one arm and said at least one receptacle. In some implementations, said interior of said at least one receptable comprises a width and a height; and said at least one arm comprises a width and a height. In some implementations, at least one of: the width of said at least one arm is less than the width of said interior of said at least one receptacle; and the height of said at least one arm is less than the height of said interior of said at least one receptacle. In some implementations, the width of said at least one arm is less than the width of said interior of said at least one receptacle, and wherein the height of said at least one arm is less than the height of said interior of said at least one receptacle.

In some implementations, said first portion comprises a frame and a membrane coupled to the frame, said membrane configured to secure to said one or more portions of the subject's skin, said frame comprising said at least one receptacle. In some implementations, said membrane comprises an opening, and wherein the membrane is configured to be secured to the subject's skin such that the catheter insertion site is positioned within said opening, and wherein said frame is coupled to the membrane along at least a portion of a perimeter of said opening. In some implementations, said at least one receptacle comprises a first receptacle and a second receptacle, and wherein said first and second receptacles extend from portions of the frame toward one another. In some implementations, said first and second receptacles are substantially aligned with one another. In some implementations, interiors of said first and second receptacles comprise the same size and/or shape. In some implementations, said frame is spaced inward from an exterior perimeter of said membrane. In some implementations, said frame comprises a more rigid material than said membrane.

In some implementations, said body comprises a C-shape. In some implementations, said body is configured to surround at least about 50% of a cross-section of said catheter hub when the catheter stabilization device is in use. In some implementations, said body is shaped to secure around a portion of a cylindrical portion of a catheter hub. In some implementations, said body is shaped to surround less than an entire perimeter of a circular cross-section of a catheter hub.

In some implementations, said at least one arm comprises two arms, and wherein said at least one receptacle comprises two receptacles. In some implementations, said two arms extend outward from said body in opposite directions. In some implementations: the two receptacles are substantially aligned with one another along a first axis, and wherein said two arms are configured to move within said two receptacles. In some implementations, said two arms are configured to move within said two receptacles: along said first axis; along a second axis that is substantially perpendicular to said first axis; and/or a third axis that is substantially parallel to said first axis. In some implementations, a first axis extends through a center of a cross-section of said at least one arm, and wherein said at least one arm is configured to rotate within said interior of said at least one receptacle about said first axis. In some implementations, said at least one arm is configured to rotate within said interior of said at least one receptacle between about 5 degrees and about 45 degrees relative to said first axis.

In some implementations: said first portion comprises at least one arm; said second portion comprises a body and at least one receptacle connected to said body and having an interior, said body configured to secure to said catheter hub, said interior of said at least one receptable configured to receive at least a portion of said at least one arm; and said first and second portions of the catheter stabilization device are movably coupled to one another via said at least one arm and said at least one receptacle. In some implementations: said interior of said at least one receptable comprises a width and a height; and said at least one arm comprises a width and a height. In some implementations, at least one of: the width of said at least one arm is less than the width of said interior of said at least one receptacle; and the height of said at least one arm is less than the height of said interior of said at least one receptacle. In some implementations, the width of said at least one arm is less than the width of said interior of said at least one receptacle, and wherein the height of said at least one arm is less than the height of said interior of said at least one receptacle. In some implementations, said first portion comprises a frame and a membrane coupled to the frame, said membrane configured to secure to said one or more portions of the subject's skin, said frame comprising said at least one arm. In some implementations, said membrane comprises an opening, and wherein the membrane is configured to be secured to the subject's skin such that the catheter insertion site is positioned within said opening, and wherein said frame is coupled to the membrane along at least a portion of a perimeter of said opening. In some implementations, said at least one arm comprises a first arm and a second arm, and wherein said first and second arms extend from portions of the frame toward one another and/or partially across said opening. In some implementations, said first and second arms are substantially aligned with one another. In some implementations, said first and second arms comprise the same size and/or shape.

In some implementations, said frame is spaced inward from an exterior perimeter of said membrane. In some implementations, said frame comprises a more rigid material than said membrane. In some implementations, said body comprises a C-shape. In some implementations, said body is configured to surround at least about 50% of a cross-section of said catheter hub when the catheter stabilization device is in use. In some implementations, said body is shaped to secure around a portion of a cylindrical portion of a catheter hub. In some implementations, said body is shaped to surround less than an entire perimeter of a circular cross-section of a catheter hub.

In some implementations, said at least one arm comprises two arms, and wherein said at least one receptacle comprises two receptacles. In some implementations, said two receptacles extend outward from said body in opposite directions. In some implementations, the two arms are substantially aligned with one another along a first axis, and wherein said two receptacles are configured to move relative to said two arms: along said first axis; along a second axis that is substantially perpendicular to said first axis; and/or a second axis that is substantially parallel to said first axis. In some implementations, a first axis extends through a center of a cross-section of said at least one receptacle, and wherein said at least one receptacle is configured to rotate about said first axis when said at least one arm is positioned within said interior of said at least one receptacle. In some implementations, said at least one receptacle is configured to rotate between about 5 degrees and about 45 degrees relative to said first axis when said at least one arm is positioned within said interior of said at least one receptacle.

In some implementations, the catheter stabilization device further comprises a third portion that is configured to enclose the catheter insertion site. Such third portion can comprise any of the covers disclosed herein. In some implementations, said third portion comprises: any of the covers disclosed herein; and any of the extension sets disclosed herein. In some implementations, said third portion is configured to removably connect to said first portion and/or said second portion.

Disclosed herein is a cover configured to at least partially enclose a catheter insertion site on a subject, the cover comprising an opening configured to provide visibility of said catheter insertion site when the cover is in use, said cover further comprising a magnifying lens positioned within said opening. Said opening can be positioned above (for example, aligned with) the catheter insertion site when the cover is secured to the subject. Such cover can be secured to the subject via a strap (for example, wrapped around a portion of the subject's body) or an adhesive attachment. Such cover can comprise any of the features described herein with respect to any of the covers disclosed herein. A catheter stabilization device can comprise such cover along with any of the hubs disclosed herein and/or any of the extension sets disclosed herein. A catheter stabilization device can comprise such cover along with a hub that is configured to at least partially secure a catheter hub connected to a catheter when the catheter is inserted at said catheter insertion site.

Disclosed herein is a catheter stabilization device comprising: a first portion configured to secure to one or more portions of the subject's skin; a second portion configured to secure to a catheter hub coupled to a catheter that is configured for insertion at said catheter insertion site; a third portion that is removably connectable to at least one of said first portion and said second portion, wherein said third portion comprises any of the covers described above or elsewhere herein. The first and second portions of the catheter stabilization device can be movably coupled to one another to allow for relative movement between said first portion of the catheter stabilization device and said catheter hub when said catheter stabilization device is in use and said catheter is inserted at said catheter insertion site.

Disclosed herein is a device configured to secure to one or more portions of skin of a subject and secure a medical component to the subject's skin such that relative movement between the medical component and the device is permitted. In some implementations, the device comprises: a first portion configured to secure to said one or more portions of the subject's skin; and a second portion configured to secure to the medical component; wherein said first and second portions of the device are movably coupled to one another to allow for relative movement between said first portion of the device and said medical component when said device is in use. In some implementations, the medical component comprises a tube, wire, or a cable. In some implementations: said device is a catheter stabilization device; said second portion is configured to secure to a catheter hub coupled to a catheter that is configured for insertion at a catheter insertion site; and wherein said first and second portions of the catheter stabilization device are movably coupled to one another to allow for relative movement between said first portion of the catheter stabilization device and said catheter hub.

Disclosed herein is an electronic device configured to connect (for example, removably connect) to a catheter stabilization device that is configured for use at a catheter insertion site on a subject, the electronic device comprising at least one of: a camera configured to generate visual signals based on the catheter insertion site and/or portions of the subject's skin proximate said catheter insertion site; one or more LEDs configured to illuminate said catheter insertion site and/or portions of the subject's skin proximate said catheter insertion site; and a sensor for measuring one or more physiological parameters of the subject.

In some implementations, the electronic device further comprises a battery. In some implementations, said one or more LEDs comprises: one or more wide spectrum LEDs configured to emit light having wavelengths between a first range; one or more narrow spectrum LEDs configured to emit light having wavelengths between a second range that is less than said first range; and one or more UV LEDs. In some implementations, said first range is between about 380 nm and about 740 nm. In some implementations, said one or more UV LEDs comprise at least one of: one or more UVA LEDs; one or more UVB LEDs; and one or more UVC LEDs. In some implementations: said one or more UVA LEDs are configured to emit light at wavelengths between about 320 nm and about 400 nm; said one or more UVB LEDs are configured to emit light at wavelengths between about 290 nm and about 320 nm; and/or said one or more UVC LEDs are configured to emit light at wavelengths between about 200 nm and about 280 nm. In some implementations, the electronic device further comprises a communication module configured to transmit said generated visual signals to an external device. In some implementations, said communication module is configured to wirelessly transmit said generated visual signals to said external device. In some implementations, the electronic device further comprises a connector port configured to connect to an external device to facilitate said transmission of said generated visual signals from said communication module to said external device. In some implementations, the electronic device further comprises one or more processors configured to: receive said generated visual signals from said camera; and determine a condition of said catheter insertion site and/or portions of the subject's skin proximate said catheter insertion site based on said received visual signals. In some implementations, said one or more processors are configured to: receive one or more visual signals generated by said camera at a first time period; receive one or more visual signals generated by said camera at a second time period that is after said first time period; compare said one or more visual signals generated by said camera at said first time period to said one or more visual signals generated by said camera at said second time period; and determine said condition of said catheter insertion site and/or portions of the subject's skin proximate said catheter insertion site based at least on said comparison.

Disclosed herein is a system including the electronic device according to any of the implementations described above and further comprising an external device, wherein the electronic device is configured to transmit said generated visual signals to said external device, and wherein said external device comprises one or more processors configured to: receive one or more visual signals generated by said camera at a first time period; receive one or more visual signals generated by said camera at a second time period that is after said first time period; compare said one or more visual signals generated by said camera at said first time period to said one or more visual signals generated by said camera at said second time period; and determine a condition of said catheter insertion site and/or portions of the subject's skin proximate said catheter insertion site based at least on said comparison.

Disclosed herein is a system including the electronic device according to any of the implementations described above and further comprising a catheter stabilization device, such as any of the catheter stabilization devices disclosed herein. In some implementations, said electronic device comprises one or more pins and said catheter stabilization device comprises one or more cavities configured to receive said one or more pins, and wherein said electronic device is configured to removably connect to said catheter stabilization device via engagement between said one or more pins and said one or more cavities. In some implementations, said catheter stabilization device comprises one or more pins and said electronic device comprises one or more cavities configured to receive said one or more pins, and wherein said electronic device is configured to removably connect to said catheter stabilization device via engagement between said one or more pins and said one or more cavities. In some implementations, said catheter stabilization device comprises a cover, said cover being any of the covers disclosed herein. In some implementations, said cover comprises a transparent material, and wherein, when said electronic device is connected to said cover, said cover is positioned between the catheter insertion site and the camera. In some implementations, said cover comprises an opening configured to provide visibility of said catheter insertion site when the cover is in use, and wherein, said electronic device is spaced from said opening when connected to said cover. In some implementations, said cover further comprises a magnifying lens positioned within said opening.

Disclosed herein is a device configured for use at a catheter insertion site on skin of a subject, the device comprising: a first portion configured to secure to the subject's skin; and a second portion configured to secure to a catheter hub coupled to a catheter that is configured for insertion at said catheter insertion site; wherein said first and second portions are movably coupled to one another to allow for relative movement between said first portion and said catheter hub when said catheter is inserted at said catheter insertion site.

In some implementations, the first and second portions of the device are coupled to one another via a gimbal arrangement. In some implementations, the second portion is rotatable relative to the first portion. In some implementations, the second portion is configured to rotate relative to the first portion at least partially about at least one of three axes, said three axes being perpendicular to one another. In some implementations, the second portion is configured to rotate relative to the first portion at least partially about at least two of said three axes. In some implementations, the second portion is configured to rotate relative to the first portion at least partially about each of said three axes.

In some implementations, the second portion comprises a ball and the first portion comprises a cavity configured to receive said ball, and wherein the ball is rotatable within said cavity. In some implementations, said cavity is configured to inhibit the ball from being removed from the cavity. In some implementations, said ball is rotatable within said cavity at least partially about each of three axes, said three axes being perpendicular to one another. In some implementations, said first and second portions are configured such that said ball is rotatable within said cavity only partially about each of said three axes. In some implementations, when the catheter hub is secured to the second portion of the device and the first portion of the device is secured to the subject's skin, the catheter hub is positioned closer to the subject's skin than the ball and/or the cavity.

In some implementations, said first portion comprises: a frame; a membrane coupled to the frame and configured to secure to the subject's skin; and a clamp coupled to the frame, wherein the clamp comprises said cavity. In some implementations, the clamp is pivotably connected to the frame. In some implementations, said membrane comprises an opening, and wherein the membrane is configured to be secured to the subject's skin such that the catheter insertion site is positioned within said opening, and wherein said frame is coupled to the membrane along at least a portion of a perimeter of said opening. In some implementations, said clamp extends across said opening of the membrane. In some implementations, said clamp extends from a first side of the frame to a second side of the frame that is opposite the first side of the frame. In some implementations, said clamp comprises: a first end configured to be connected to said first side of the frame; and a second end configured to be connected to said second side of the frame. In some implementations, said cavity is arranged between the first and second ends of the clamp. In some implementations, the first end is pivotably connected to said first side of the frame and configured to pivot between: a first position in which the second end is secured to said second side of the frame; and a second position in which the second end is not secured to said second side of the frame. In some implementations, the clamp comprises a pin at the first end, and wherein the frame is configured to pivotably secure the pin. In some implementations, the clamp comprises one or more living hinges configured to allow the clamp to pivot between said first and second positions. In some implementations, the one or more living hinges are arranged at the first end of the clamp. In some implementations, the one or more living hinges comprises a first living hinge and a second living hinge spaced from the first living hinge. In some implementations, the clamp further comprises one or more posts and the frame comprises one or more openings configured to receive the one or more posts. In some implementations, the one or more posts are arranged at the first end of the clamp and are configured to secure the first end of the clamp to the frame.

In some implementations, the second portion further comprises at least one protrusion extending outward from the ball, and wherein the clamp further comprises at least one slot configured to receive said at least one protrusion. In some implementations, the at least one slot is configured to limit rotation of the ball via contact with the at least one protrusion. In some implementations, the at least one slot is configured to limit a degree of rotation of the ball about a first axis that extends through a center of said cavity. In some implementations, said first portion is configured to limit a degree of rotation of the ball about a second axis that is perpendicular to said first axis. In some implementations, the second portion is a catheter hub holder comprising: a body configured to secure to said catheter hub; a stem having a first end connected to the body and a second end opposite the first end of the stem; and said ball, wherein said ball is connected to the second end of the stem. In some implementations, said body comprises two curved legs having cantilevered ends that are spaced from one another, and wherein said second portion further comprises a pair of arms extending from each of said cantilevered ends away from one another, said pair of arms configured to facilitate securement of the catheter hub within said body. In some implementations, the device further comprises a cover configured to removably secure to the first portion and at least partially enclose the catheter insertion site.

In some implementations, the device further comprises a fluid tube assembly configured to deliver one or more fluids to the catheter. In some implementations, the fluid tube assembly comprises: a first connector configured to connect to said catheter hub; a first tube having a first end connected to said first connector and a second end opposite the first end; a second connector connected to the second end of the first tube; and a second tube having a first end connected to the second connector and a second end opposite the first end of the second tube. In some implementations: the second connector is configured to fluidly couple the first and second tubes together and is further configured to engage a portion of the cover; when the second connector is engaged with the portion of the cover, the first tube is arranged within an interior of the cover and the second tube is arranged outside of said interior; and the first tube comprises a flexible material that is configured to accommodate said relative movement between said first portion and said catheter hub when said first connector is connected to said catheter. In some implementations: the first tube is more flexible than the second tube; the second tube is more rigid than the first tube; and/or the second tube is more durable than the first tube.

Disclosed herein is an assembly for delivering fluid to a subject via a catheter inserted at a catheter insertion site, the assembly comprising: a cover configured to be placed over the catheter insertion site; and a fluid delivery assembly configured to be connected to the cover. In some implementations, the fluid delivery assembly comprises: a first tube configured to be connected to a catheter hub connected to the catheter; and a second tube connected to the first tube. In some implementations, the fluid delivery assembly is configured such that: when the fluid delivery assembly is connected with the cover, the first tube extends within an interior of the cover and the second tube extends outside of said interior; and the first tube comprises a flexible material that is configured to accommodate movement of said catheter hub when said first tube is connected to said catheter.

In some implementations, the fluid delivery assembly further comprises a first connector configured to connect the first and second tubes to one another, wherein the first connector is configured to engage a portion of the cover. In some implementations, when the first connector is engaged with the portion of the cover, the first tube extends within said interior of the cover and the second tube extends outside of said interior. In some implementations, the fluid delivery assembly further comprises a second connector configured to connect the first tube to said catheter hub. In some implementations, the fluid delivery assembly further comprises a third connector configured to secure the second connector to the portion of the cover. In some implementations, the third connector secures (for example, threadably secures) to the portion of the cover and/or a portion of the second connector. In some implementations, a portion of the second connector is received by a portion of the third connector. In some implementations: the first tube is more flexible than the second tube; the second tube is more rigid than the first tube; and/or the second tube is more durable than the first tube. In some implementations, the assembly further comprises a hub configured to secure to the subject's skin and secure to said catheter hub. Such hub can comprise any of the hubs disclosed herein.

Disclosed herein is a device configured for use at a catheter insertion site of a subject, the device comprising: a base comprising an opening, the base configured to be secured to skin of the subject such that said opening is positioned over the catheter insertion site; and a catheter hub holder configured to secure to a catheter hub connected to a catheter that is configured for insertion at the catheter insertion site; wherein the catheter hub holder is movably coupled to the base to allow for relative movement between the base and the catheter hub and catheter when said catheter is inserted at the catheter insertion site.

In some implementations, the catheter hub holder is movably coupled to the base via a gimbal arrangement. In some implementations, wherein the catheter hub holder is rotatably coupled to the base and configured to rotate at least partially about three, mutually orthogonal axes. In some implementations, the catheter hub holder comprises a ball and the base comprises a cavity configured to receive said ball, and wherein said ball is rotatable within said cavity. In some implementations, the catheter hub holder further comprises: a body configured to surround and secure to a portion of said catheter hub; and a stem connected to and extending between the body and said ball such that the ball is spaced from the body. In some implementations, the body is configured to hold said catheter hub above skin of the subject when the device is in use. In some implementations, the base further comprises a membrane configured to secure to the subject's skin and a frame connected to the membrane, the frame being more rigid than the membrane, and wherein the catheter hub holder is movably coupled to said frame. In some implementations, the base further comprises a clamp connected to the frame, the clamp comprising said cavity of the base, and wherein the catheter hub holder is movably coupled to the frame via the clamp. In some implementations, at least one of the catheter hub holder and the clamp is configured such that rotation of the catheter hub holder relative to the clamp is limited with respect to at least one of said three, mutually orthogonal axes. In some implementations: the clamp comprises at least one slot; the catheter hub holder further comprises at least one ear extending outward from the ball and positioned within the at least one slot of the clamp; and contact between portions of the clamp forming said at least one slot and the at least one ear limit an amount of rotation of the catheter hub holder relative to the clamp about at least one of said three, mutually orthogonal axes. In some implementations: the membrane comprises said opening of the base; the frame is connected to the membrane along a perimeter of said opening; the clamp comprises a first end and a second end, the first end being pivotably connected to a first portion of the frame such that the clamp is pivotable between a closed position and an open position; in said closed position, the second end of the clamp is connected to a second portion of the frame; and in said open position, the second end of the clamp is not connected to the second portion of the frame. In some implementations, the clamp comprises a pin at the first end, and wherein the first portion of the frame is configured to pivotably secure the pin. In some implementations, the clamp comprises one or more living hinges at the first end configured to allow the clamp to pivot between said open and closed positions. In some implementations, the clamp further comprises a latch at the second end that is configured to removably connect to one or more protrusions arranged at the second portion of the frame. In some implementations, the device further comprises a cover configured to removably connect to the base and enclose the opening and the catheter insertion site.

### BRIEF DESCRIPTION OF THE DRAWINGS

Certain features of this disclosure are described below with reference to the drawings. The illustrated embodiments are intended to illustrate, but not to limit the embodiments. Various features of the different disclosed embodiments can be combined to form further embodiments, which are part of this disclosure.

FIGS. 1A-1B illustrate top perspective views of a catheter stabilization device in accordance with aspects of this disclosure.

FIG. 1C illustrates a bottom perspective view of the catheter stabilization device of FIGS. 1A-1B in accordance with aspects of this disclosure.

FIGS. 1D-1E illustrate top and bottom views of the catheter stabilization device of FIGS. 1A-1B in accordance with aspects of this disclosure.

FIG. 1F illustrates a cross-sectional view taken through a portion of the catheter stabilization device of FIGS. 1A-1B in accordance with aspects of this disclosure.

FIG. 1G illustrates an exploded view of the catheter stabilization device of FIGS. 1A-1B in accordance with aspects of this disclosure.

FIGS. 1H-1K illustrate various views of a cover of the catheter stabilization device of FIGS. 1A-1B in accordance with aspects of this disclosure.

FIG. 1L illustrates an exploded view of an extension set of the catheter stabilization device of FIGS. 1A-1B in accordance with aspects of this disclosure.

FIGS. 1M-1P illustrate various perspective views of a hub of the catheter stabilization device of FIGS. 1A-1B in accordance with aspects of this disclosure.

FIGS. 1Q-1R illustrate bottom and top views of the hub of FIGS. 1M-1P in accordance with aspects of this disclosure.

FIGS. 1S-1U illustrate cross-sectional views taken through the portions of the hub of FIGS. 1M-1P in accordance with aspects of this disclosure.

FIGS. 1V-1Y illustrate implementations of catheter hub holders in accordance with aspects of this disclosure.

FIG. 1Z illustrates an enlarged view of a portion of the hub of FIGS. 1M-1P in accordance with aspects of this disclosure.

FIGS. 1AA-1BB illustrate cross-sectional views of portions of another implementation of a hub in accordance with aspects of this disclosure.

FIGS. 2A-2D illustrate an electronic device in accordance with aspects of this disclosure.

FIGS. 3A-3B illustrate an implementation of a cover that can be connected to the electronic device of FIGS. 2A-2D, and FIGS. 3C-3D illustrate such cover connected to the electronic device.

FIGS. 4A-4B illustrate top perspective views and FIG. 4C illustrates a bottom perspective view of another implementation of a catheter stabilization device in accordance with aspects of this disclosure.

FIGS. 4D-4E illustrate top and bottom views of the catheter stabilization device of FIGS. 4A-4B in accordance with aspects of this disclosure.

FIG. 4F illustrates a cross-sectional view taken through a portion of the catheter stabilization device of FIGS. 4A-4D in accordance with aspects of this disclosure.

FIG. 4G illustrates an exploded view of the catheter stabilization device of FIGS. 4A-4D in accordance with aspects of this disclosure.

FIGS. 4H-4Q illustrate various views of a cover of the catheter stabilization device of FIGS. 4A-4D in accordance with aspects of this disclosure.

FIGS. 4R-4V illustrate portions of an extension set of the catheter stabilization device of FIGS. 4A-4D in accordance with aspects of this disclosure.

FIGS. 4W-4Z illustrate various top perspective views of a hub of the catheter stabilization device of FIGS. 4A-4D in accordance with aspects of this disclosure.

FIGS. 4AA-4BB illustrate top and bottom views of the hub of FIGS. 4W-4Z in accordance with aspects of this disclosure.

FIGS. 4CC-4DD illustrate side views of the hub of FIGS. 4W-4Z in accordance with aspects of this disclosure.

FIGS. 4EE-4FF illustrate front and rear views of the hub of FIGS. 4W-4Z in accordance with aspects of this disclosure.

FIG. 4GG illustrates a cross-sectional view taken through a portion of the hub of FIGS. 4W-4Z in accordance with aspects of this disclosure.

FIGS. 4HH-4KK illustrate various portions of the hub of FIGS. 4W-4Z in accordance with aspects of this disclosure.

FIGS. 4LL-4NN illustrate an implementation of a catheter hub holder in accordance with aspects of this disclosure.

FIGS. 4OO-4RR illustrate an implementation of a clamp in accordance with aspects of this disclosure.

FIGS. 4SS-4TT illustrate cross-sectional views taken through portions of the hub of FIGS. 4W-4Z in accordance with aspects of this disclosure.

FIG. 5A illustrates a top perspective view of another implementation of a catheter stabilization device in accordance with aspects of this disclosure.

FIG. 5B illustrates a top view of the catheter stabilization device of FIG. 5A in accordance with aspects of this disclosure.

FIG. 5C illustrates a cross-sectional view taken through a portion of the catheter stabilization device of FIGS. 5A-5B in accordance with aspects of this disclosure.

FIG. 5D illustrates an enlarged view of a portion of that which is shown in FIG. 5C.

FIGS. 5E-5F illustrate top perspective views of a cover of the catheter stabilization device of FIG. 5A in accordance with aspects of this disclosure.

FIG. 5G illustrates an extension set of the catheter stabilization device of FIG. 5A in accordance with aspects of this disclosure.

FIG. 5H illustrates a portion of the extension set of FIG. 5G in accordance with aspects of this disclosure.

FIG. 6A illustrates a top perspective view of another implementation of a catheter stabilization device in accordance with aspects of this disclosure.

FIG. 6B illustrates a top view of the catheter stabilization device of FIG. 6A in accordance with aspects of this disclosure.

FIG. 6C illustrates a cross-sectional view taken through a portion of the catheter stabilization device of FIGS. 6A-6B in accordance with aspects of this disclosure.

FIGS. 6D-6F illustrate various portions of an extension set of the catheter stabilization device of FIGS. 6A-6B in accordance with aspects of this disclosure.

FIG. 6G illustrates an enlarged view of a portion of that which is shown in FIG. 6C.

FIGS. 7A-7D illustrate top perspective views of an implementation of a hub in accordance with aspects of this disclosure.

FIGS. 7E-7F illustrate top and bottom views of the hub of FIGS. 7A-7D in accordance with aspects of this disclosure.

FIGS. 7G-7H illustrate side views of the hub of FIGS. 7A-7D in accordance with aspects of this disclosure.

FIGS. 7I-7J illustrate front and rear views of the hub of FIGS. 7A-7D in accordance with aspects of this disclosure.

FIGS. 7K-7Q illustrate various views of portions of the hub of FIGS. 7A-7D in accordance with aspects of this disclosure.

FIGS. 7R-7U illustrate an implementation of a clamp in accordance with aspects of this disclosure.

FIGS. 8A-8B illustrate top perspective views of another implementation of a hub in accordance with aspects of this disclosure.

FIG. 8C illustrates an exploded perspective view of portions of the hub of FIGS. 8A-8B in accordance with aspects of this disclosure.

FIG. 8D illustrates an enlarged view of a portion of the hub of FIGS. 8A-8B in accordance with aspects of this disclosure.

FIGS. 8E-8F illustrate an implementation of a catheter hub holder in accordance with aspects of this disclosure.

FIGS. 8G-8J illustrate an implementation of a clamp in accordance with aspects of this disclosure.

### DESCRIPTION

Various features and advantages of the devices, systems, and methods of the technology described herein will become more fully apparent from the following description of the examples illustrated in the figures. These examples are intended to illustrate the principles of this disclosure, and this disclosure should not be limited to merely the illustrated examples. The features of the illustrated examples can be modified, combined, removed, and/or substituted as will be apparent to those of ordinary skill in the art upon consideration of the principles disclosed herein.

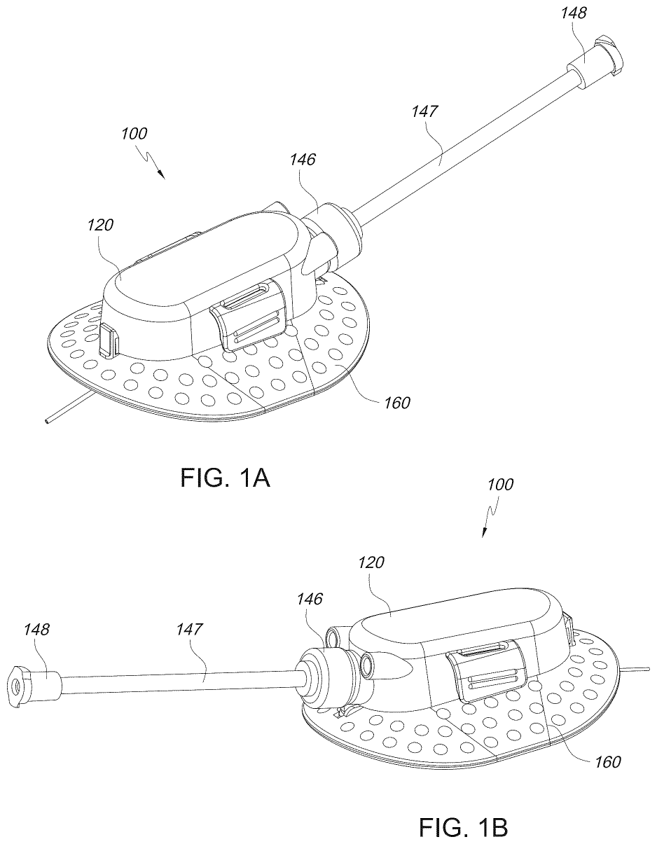

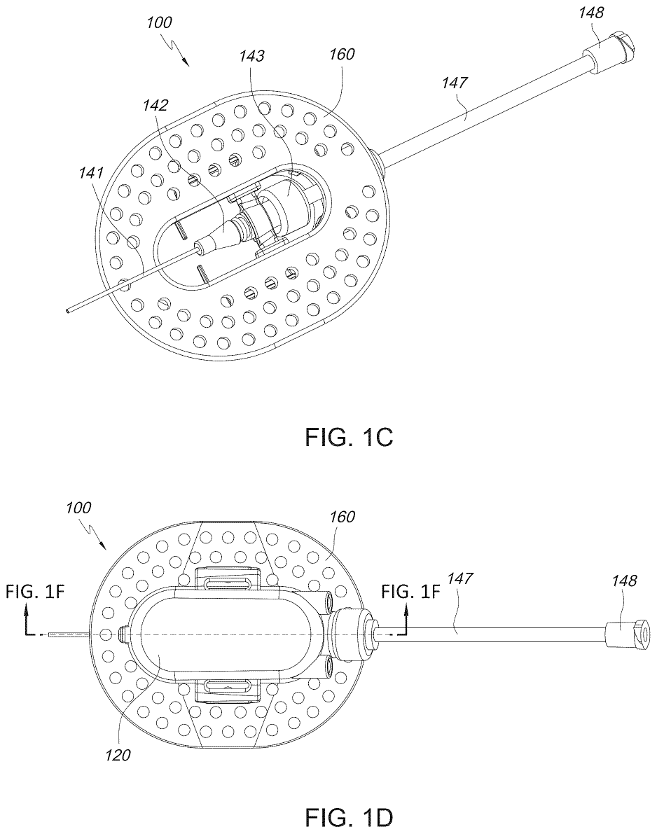

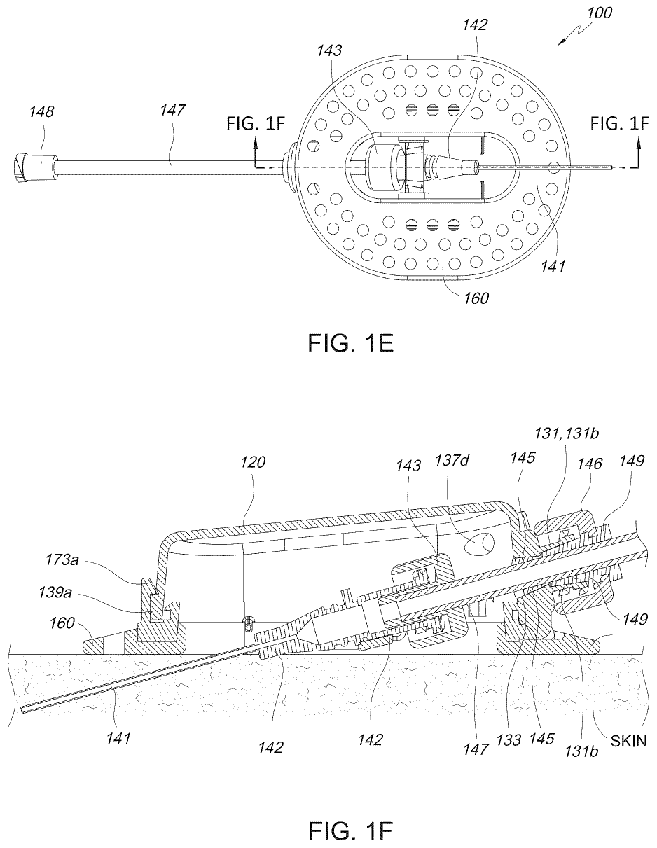

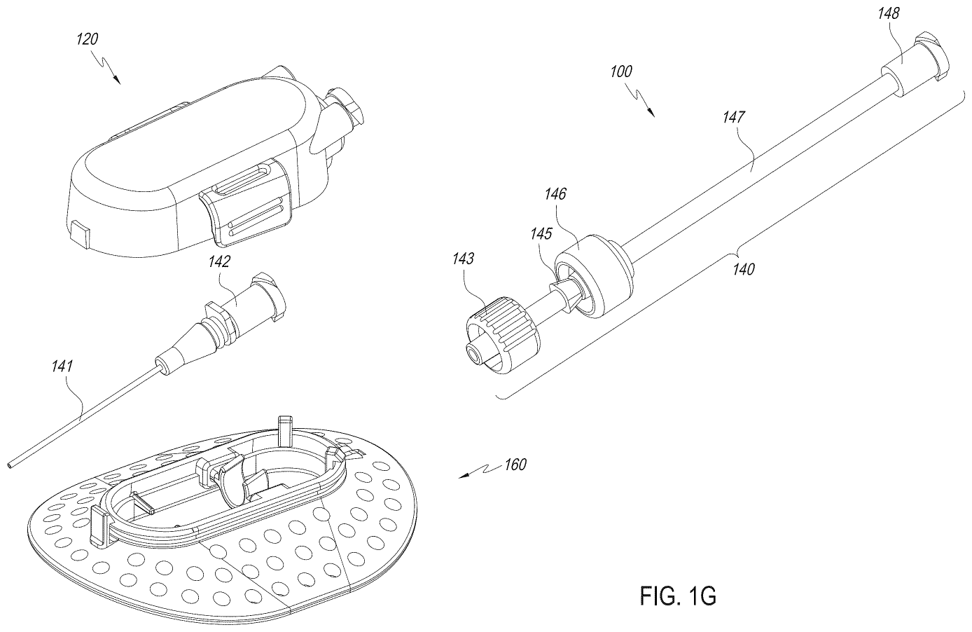

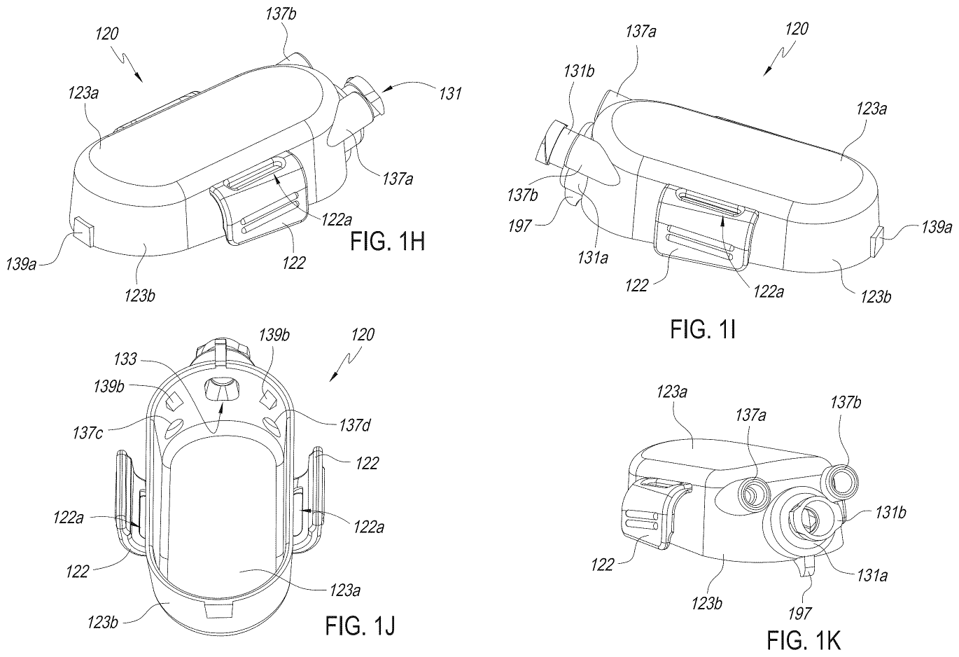

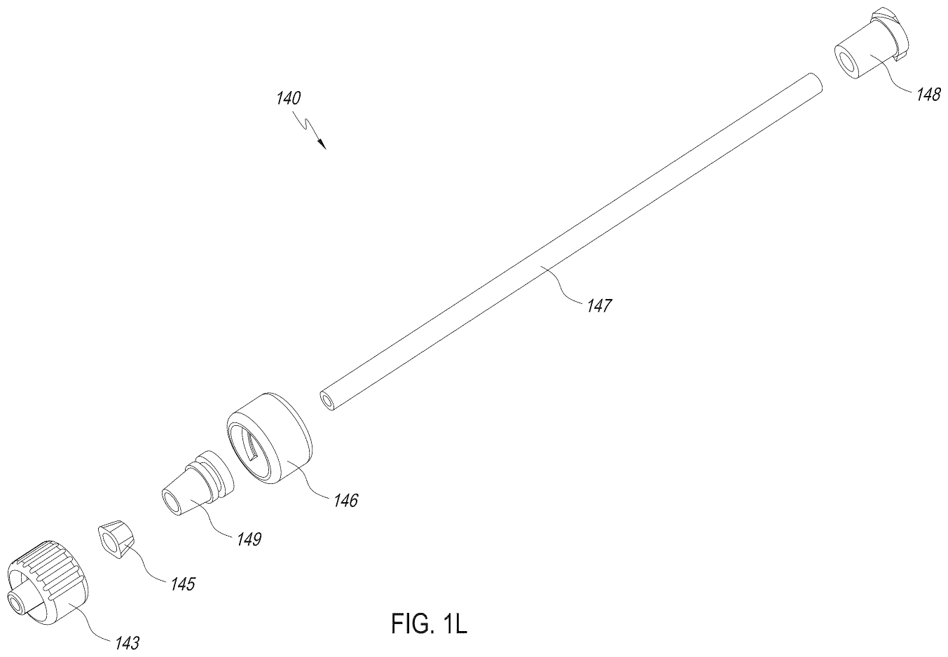

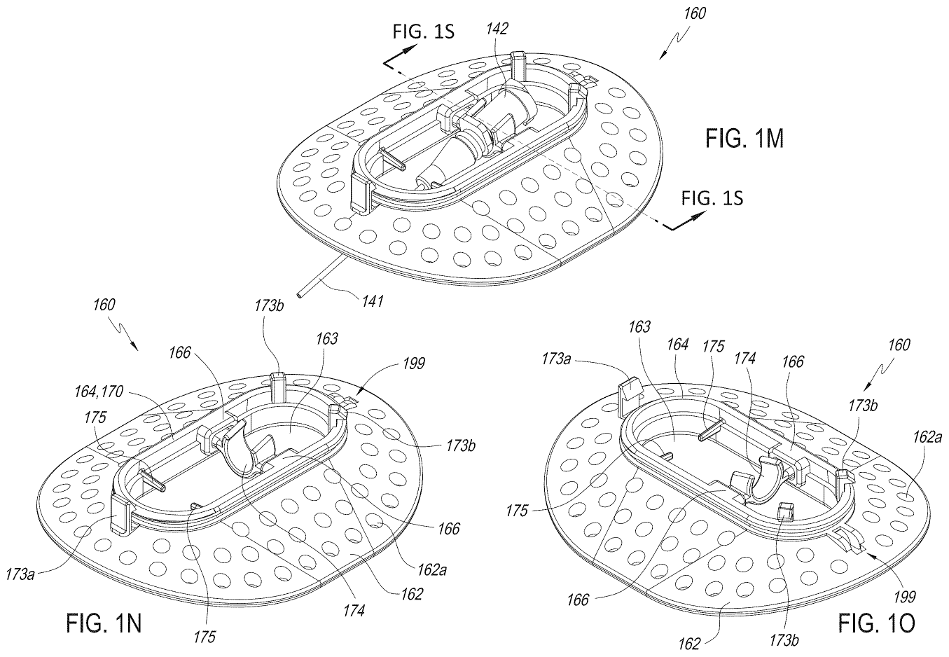

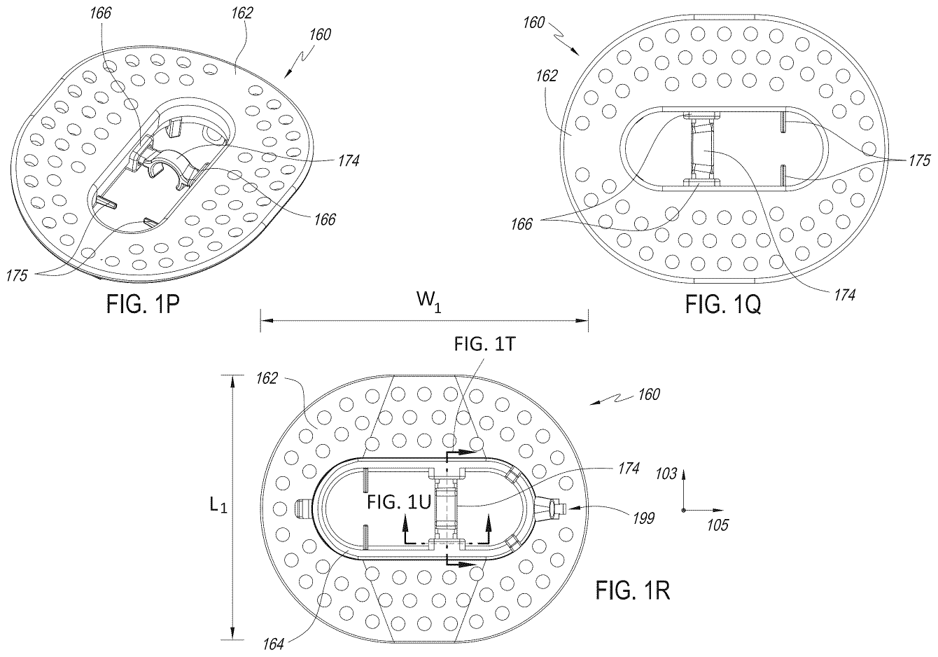

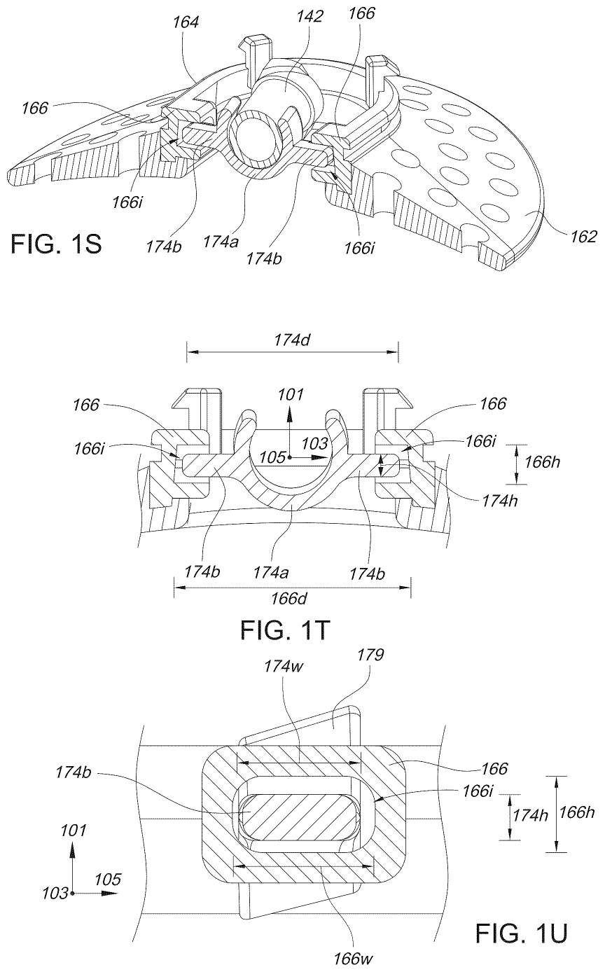

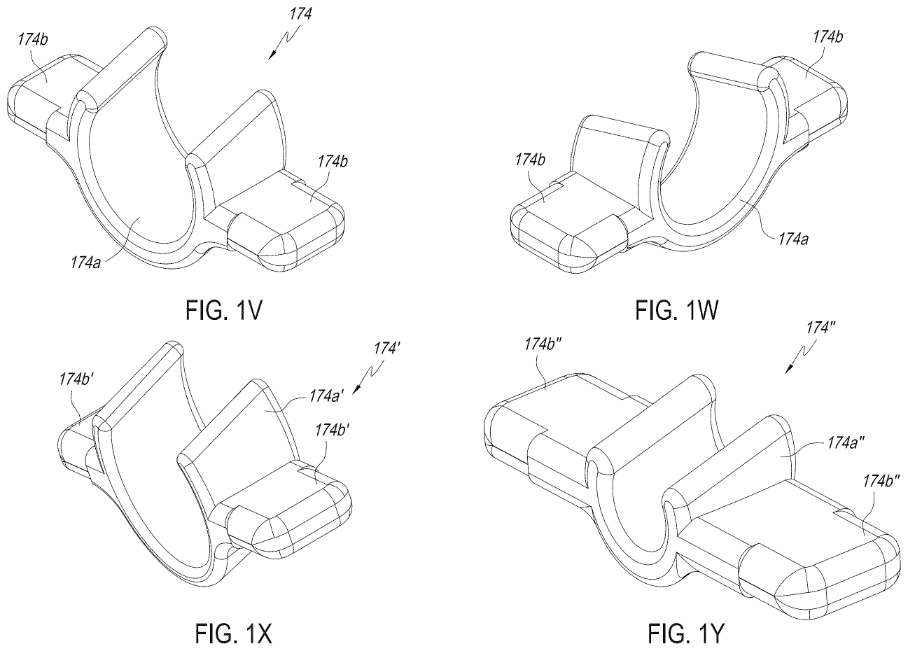

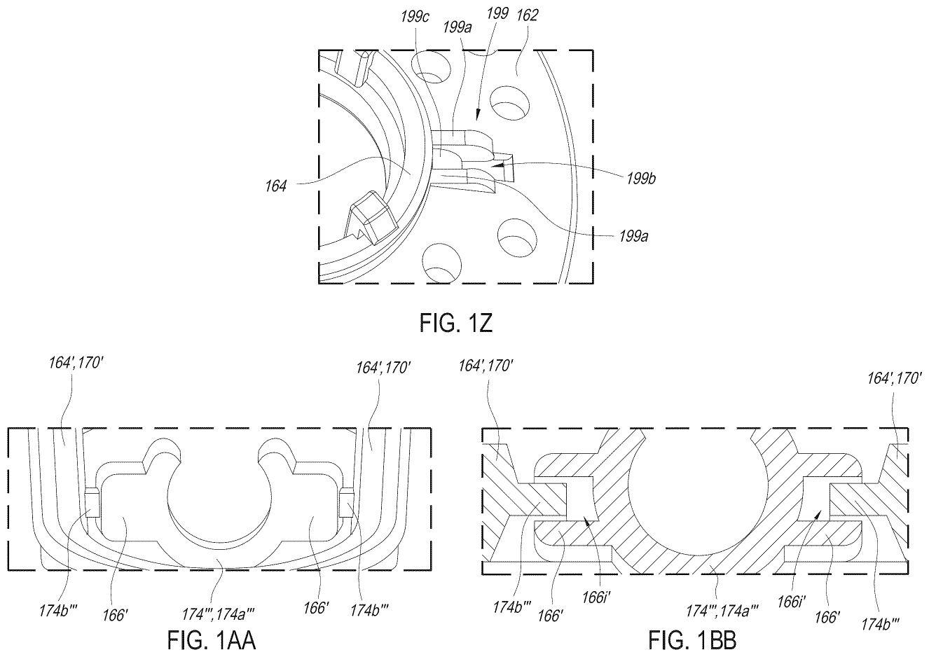

FIGS. 1A-1F illustrate a catheter stabilization device 100 (which may also be referred to as a “catheter housing”, “catheter housing device”, “catheter securement device”, “catheter stabilization system”, or “device”). FIGS. 1A-1B illustrate top perspective views, FIG. 1C illustrates a bottom perspective view, and FIGS. 1D-1E illustrate top and bottom views (respectively) of catheter stabilization device 100. FIG. 1G illustrates an exploded view of an implementation of catheter stabilization device 100 proximate a catheter 141 and a catheter hub 142 that is connected to catheter 141. Catheter 141 (which may also be referred to as a “catheter cannula”) can be configured to be inserted into a subject's vein, for example, with the use of a needle that can be coupled (for example, retractably coupled) to catheter hub 142 as will be understood by one of skill in the art. FIG. 1F illustrates a cross-section taken through a portion of catheter stabilization device 100 (as indicated in FIGS. 1E-1F) in a condition of use where device 100 is secured to skin of a subject. Although not illustrated in FIG. 1F, it is to be understood that catheter 141 can be at least partially positioned within a vein of the subject when in use. With reference to FIG. 1G, catheter stabilization device 100 can include a cover 120 and a hub 160. In some implementations, catheter stabilization device 100 includes an extension set 140. In some implementations, cover 120 and extension set 140 are coupled to one another, for example, in a similar manner as described with respect to any of the covers and extension sets in jointly owned PCT. Pub. No. WO2022/140633A1, the entirety of which is incorporated by reference herein. In some variants, catheter stabilization device 100 includes hub 160 but does not include cover 120 and/or does not include extension set 140. In some variants, catheter stabilization device 100 includes cover 120 but does not include hub 160 and/or does not include extension set 140.

FIGS. 1H-1I and 1K illustrate top perspective views of cover 120 and FIG. 1J illustrates a bottom perspective view of cover 120. Cover 120 can be similar or identical to any of the covers disclosed in PCT. Pub. No. WO2022/140633A1 in some or many respects. Cover 120 can include a top portion 123a and a wall 123b that extends outward from the top portion 123a. Wall 123b can extend transverse (for example, generally perpendicular to) such top portion 123a and/or can define a perimeter of the cover 120. Cover 120 can be made of a variety of materials, including plastic, such as polyphenyl ether (PPE), polycarbonate (PC), among others. In some implementations, cover 120 is transparent or semi-transparent. In some implementations, cover 120 is made of a more rigid material than hub 160 or portions of hub 160. For example, in some implementations, cover 120 is made of a more rigid material than membrane 162 of hub 160. In some implementations, cover 120 includes one or more wings 122 which can extend outward from wall 123b and/or top portion 123a. Wing(s) 122 can extend outward from the wall 123b and/or top portion 123a and can be utilized to secure tube 147 when in use. Wings 122 can include openings 122a (which may also be referred to as “slots”) that can allow a portion of the tube 147 to be viewed and/or can receive a portion of a strap that may be used to secure cover 120 (and/or hub 160 and extension set 140, if connected to cover 120) to a subject, for example, by wrapping around the subject's arm. In some implementations wings 122 include one or more ribs to aid a user in gripping and/or handling the cover 120.

In some implementations, cover 120 includes protrusion 139a that is configured to engage latch arm 173a of hub 160. Additionally or alternatively, cover 120 can include protrusion(s) 139b which can be configured to engage latch arms 173b of hub 160. Engagement between protrusions 193a, 139b and latch arms 173a, 173b can allow cover 120 and hub 160 to secure to one another.

Cover 120 can include a connector 131 configured to facilitate connection to extension set 140. Connector 131 can extend from wall 123b of cover 120 around opening 133. Connector 131 can be secured to connector 146 of extension set 140. In some implementations: when connectors 131, 146 are connected to one another, movement (for example, sliding) of cover 120 and/or connector 146 relative to tube 147 is inhibited (for example, prevented); and when connectors 131, 146 are disconnected from one another, movement (for example, sliding) of cover 120 and/or connector 146 relative to tube 147 can be permitted. Connector 131 can include a connector body 131a extending outward from wall 123b and a threaded portion 131b extending outward from connector body 131a. Connector body 131a and/or threaded portion 131b can have a generally cylindrical shape and have an opening aligning with opening 133 so as to allow tube 147 to extend therethrough.

Cover 120 can include one or both of ports 137a, 137b that can extend from a portion of cover 120, such as wall 123b (or alternatively, from top portion 123a). Ports 137a, 137b can be similar or identical to any of the ports disclosed in PCT. Pub. No. WO2022/140633A1. With reference to FIG. 1J, cover 120 can include openings 137c, 137d to facilitate fluid communication between ports 137a, 137b and an interior of cover 120 (for example, an interior defined by top portion 123a and wall 123b). As also shown in FIG. 1J, cover 120 can include an opening 133. Opening 133 can be sized and/or shaped to correspond to a size and/or shape of a stopper 145 that can be positioned around and/or secured to tube 147, as described in more detail below and as illustrated in FIG. 1F.

With reference to FIGS. 1I and 1K, in some implementations, cover 120 includes a hook 197 configured to engage with a notch 199 of hub 160 (see FIG. 1Z). Hook 197 can extend outward from a portion of cover 120, for example, from wall 123b. In some implementations, hook 197 extends outward from an edge of wall 123b. Alignment and/or positioning of hook 197 within notch 199 can help align and/or position cover 120 relative to hub 160 when being secured thereto.

Cover 120 can be coupled (for example, movably coupled) to extension set 140 in a similar or identical manner as that described in PCT. Pub. No. WO2022/140633A1 with reference to any of the cover and extension sets disclosed therein. In such implementations, in which cover 120 is configured to move (for example, slide) along tube 147, alignment and/or positioning of hook 197 within notch 199 can help align and/or position both cover 120 and extension set 140 when cover 120 is moved along tube 147 and secured to hub 160, for example, after connector 143 is secured to catheter hub 142. With reference to FIG. 1Z, in some implementations, notch 199 is formed by walls 199a spaced from one another by a gap and/or a groove 199b in a portion of membrane 162. In some implementations, notch 199 is additionally formed by a curved portion 199c of frame 164. In some implementations, hook 197 has a size and/or shape corresponding to a space defined by walls 199a, groove 199b, and/or curved portion 199c.

FIG. 1L illustrates an exploded view of extension set 140. Extension set 140 (which also may be referred to as a “fluid tube assembly”) can be utilized with and/or can interact with cover 120 and/or hub 160 in a manner that is similar to that described with respect to the extension sets in PCT. Pub. No. WO2022/140633A1. Extension set 140 can include a connector 143, a stopper 145, a gasket 149, a connector 146, a tube 147, and a connector 148. Connector 143 can be configured to secure to catheter hub 142 to facilitate fluid communication between catheter hub 142 (and connected catheter 141) and tube 147. Connector 143 can be a male luer connector. Connector 148 can be configured to connect to a fluid delivery device. Connector 148 can be a female luer connector. In some implementations, connector 146 and/or gasket 149 are the only components of extension set 140 that are movable relative to tube 147.

In some implementations, stopper 145 is permanently fixed (for example, bonded) to tube 147. Stopper 145 can have an opening sized and/or shaped to receive tube 147. In some implementations, stopper 145 has a tapered shape. In some implementations, stopper 145 has a frustoconical shape. Stopper 145 can have a size and/or shape that corresponds to a size and/or shape of opening 133 of cover 120 (see FIGS. 1F and 1J). As shown in FIG. 1F, stopper 145 can fit within opening 133 of cover 120. Stopper 145 can be positioned within opening 133 when cover 120 is secured to hub 160 and/or connector 146 (and gasket 149) is secured to connector 131 of cover 120 (for example, via threaded portion 131b). Stopper 145 can: inhibit or prevent tube 147 from being pulled out of cover 120; and/or isolate a portion of tube 147 inside cover 120, connector 143, catheter hub 142, and/or catheter 141 from forces applied to tube 147 outside cover 120. For example, if a force is applied to a portion of tube 147 outside cover 120 in a direction along a longitudinal axis of tube 147 and/or transverse (for example, perpendicular) to such axis, stopper 145 can engage wall 123b and prevent such force from causing movement of connector 143, catheter hub 142, and/or catheter 141. Stopper 145 can be utilized alone or in combination with gasket 149, which is described below. In some cases, stopper 145 can be engaged if the force applied to tube 147 exceeds a capacity of gasket 149 to resist such force.

With reference to FIGS. 1L and 1F, extension set 140 can include a gasket 149 and a connector 146. Connector 146 can be a spin lock connector configured to connect to a threaded connector, such as threaded portion 131b of cover 120. Gasket 149 (for example, a portion thereof) can be positioned within connector 146, for example, around tube 147. Gasket 149 can be operably positioned by connector 146 such that pressure is applied to gasket 149 when connectors 146, 131 are secured to one another (see FIG. 1F). When connectors 146, 131 are connected to one another, movement of connector 146 and the cover 120 along tube 147 can be inhibited (for example, prevented), via a gripping force applied by gasket 149 onto tube 147 induced by pressure applied to gasket 149 by connectors 146, 131. Such configurations can allow the connectors 146, 131 to “lock” a portion of tube 147 such that forces applied to tube 147 outside cover 120 are not translated to: a portion of tube 147 inside cover 120; connector 143; catheter hub 142; and/or catheter 141. This can advantageously inhibit dislodgement of catheter 141 and/or catheter hub 142. Gasket 149 (for example, a portion thereof) can comprise a tubular and/or frustoconical shape. Gasket 149 can be moveable (for example, slidable) along tube 147 when connector 146 is disconnected from connector 131 of cover 120. Gasket 149 can have a smaller length than tube 147. Gasket 149 can be made of a variety of materials, such as polyvinyl chloride (PVC), a latex free material, and/or other materials. Gasket 149 can comprise the same material as tube 147 or can comprise a different material than tube 147.

Catheter stabilization device 100 can be utilized at a catheter insertion site to secure catheter hub 142 when catheter 141 is inserted in the catheter insertion site. Catheter stabilization device 100 can secure to skin on a portion of a subject's body (such as an arm) and also secure to such catheter hub 142. The securement to catheter hub 142 allows catheter stabilization device 100 to stabilize (for example, operably position) catheter 141 with respect to the catheter insertion site and/or a subject's vein in which catheter 141 extends. In some cases, movement of catheter stabilization device 100 (and/or skin to which device 100 is attached) may cause catheter 141 to move relative to the catheter insertion site and/or the vein in which catheter 141 extends. As discussed previously, it may be desirable to inhibit relative movement between catheter 141 and the subject's vein and/or the insertion site. As also discussed, it may be desirable to accommodate micro-movements of catheter 141 within the vein. Catheter stabilization device 100 can include a first portion that is configured to secure to the subject's skin and a second portion that is configured to secure to catheter hub 142 connected to catheter 141. Such first and second portions can be movably coupled to one another to allow for relative movement between the first portion and catheter hub 142 and catheter 141. Membrane 162 and/or frame 164, described below, can be implementations of such first portion. Catheter hub holder 174, also described below, can be an implementation of such second portion.

FIGS. 1M-1BB illustrate hub 160 or portions thereof. Hub 160 (which also may be referred to as a “dock” or “base”) can include a frame 164 and a membrane 162. In some implementations, frame 164 comprises a material that is more rigid than membrane 162. In some implementations, membrane 162 is overmolded onto frame 164, for example, in a similar manner as that described in PCT. Pub. No. WO2022/140633A1 with respect to any of the membranes and frames disclosed therein. Frame 164 can include a frame body 170 and latch arms 173a, 173b that can be similar (respectively) to any of the frame bodies and latch arms described in PCT. Pub. No. WO2022/140633A1. Latch arms 173a, 173b can engage protrusions 139a, 139b of cover 120 in a similar manner as described above. In some implementations, membrane 162 is an adhesive layer (for example, coupled with a foam layer) that is configured to secure to skin and configured to be secured to frame 164.

Membrane 162 can include one or more or a plurality of perforations 162a (also referred to herein as “apertures”) to facilitate breathability of the subject's skin when membrane 162 is secured thereto. Membrane 162 can include an opening 163 which can be positioned over a site where a needle and/or catheter 141 is to be (or has been) inserted into a subject (for example, patient). Opening 163 can have a rounded shape. For example, opening 163 can comprise an oblong shape. In some implementations, opening 163 is not partitioned and/or divided by any portion of membrane 162. In some implementations, when catheter stabilization device 100 is assembled and secured to the subject's skin (for example, as shown in FIG. 1F), no portion of membrane 162 is positioned between: catheter hub 142 and the subject's skin; connector 143 and the subject's skin; and/or catheter hub holder 174 and the subject's skin. Membrane 162 can be made of a flexible material. Membrane 162 can be made of a different material than cover 120. Membrane 162 can comprise, for example, thermoplastic elastomers (TPE), thermoplastic polyurethane (TPU), and/or polyvinyl chloride (PVC), among other materials. In some implementations, membrane 162 comprises a material that is more flexible than a material comprised by cover 120. Membrane 162 can be more flexible than frame 164. In some implementations, membrane 162 is transparent or semi-transparent. In some variants, hub 160 does not include frame 164. In some of such variants, hub 160 can be configured to removably connect to cover 120.

Frame 164 can include one or more indicators extending from frame body 170 that can aid in positioning hub 160 relative to a catheter insertion site and/or relative to catheter 141 and/or catheter hub 142. For example, frame 164 can include fingers 175 extending outward from a surface of frame body 170. As shown, fingers 175 can extend from opposing sides of frame body 170 toward one another. Fingers 175 can be arranged closer to one of two opposite ends of hub 160 and/or frame 164. Fingers 175 can have a first end connected to a portion of frame body 170 and a second end that is “free”. The free ends of fingers 175 can be spaced from one another by a gap. Such gap can allow the catheter insertion site to be viewed and positioned therewithin, for example, when hub 160 is secured to the subject's skin. In some implementations, fingers 175 (and/or other portions of hub 160) and/or cover 120 are made of a transparent material. In some of such implementations, free ends of fingers 175 are painted with a color that more easily allows visibility of such free ends and therefore aids alignment and/or positioning of hub 160 (and fingers 175) relative to a catheter insertion site.

FIG. 1M illustrates a top perspective view of hub 160 with catheter hub 142 secured thereto. FIGS. 1N and 1O illustrate top perspective views, FIG. 1P illustrates a bottom perspective view, FIG. 1Q illustrates a bottom view, and FIG. 1R illustrates a top view (respectively) of hub 160 without catheter hub 142 and catheter 141. As mentioned previously, hub 160 can include frame 170 and membrane 162. Hub 160 can further include a catheter hub holder 174 (which may also be referred to as a “catheter hub retainer”). Frame 164 and/or membrane 162 can be configured to secure (for example, removably secure) to the subject's skin and catheter hub holder 174 can be configured to secure to catheter hub 142.

With reference to FIGS. 1M-1R, frame 164 can include one or more receptacles 166 (for example, two receptables). As mentioned above, frame 164 can include a frame body 170. In such implementations, receptacle(s) 166 can extend from frame body 170. In some implementations, frame body 170 comprises an annular shape. In some implementations, frame 164 includes two receptacles 166 extending outward from opposite sides (or portions) of frame body 170 toward one another and/or across an opening defined by frame 164 and/or opening 163 in membrane 162. With reference to FIGS. 1V-1W, catheter hub holder 174 can include a body 174a and arms 174b extending outward from body 174a (for example, in opposite directions from one another). Each of arms 174b can have a first end connected to body 174a and a second end (which may be referred to as a “free end”) opposite such first end. In some implementations, no portion of catheter hub holder 174 contacts skin of the subject when catheter stabilization device 100 (for example, frame 164 and/or membrane 162) is secured to the subject. For example, in some implementations, when catheter stabilization device 100 (for example, frame 164 and/or membrane 162) is secured to the subject, body 174b does not contact skin of the subject and/or arms 174a do not contact skin of the subject.

FIGS. 1S-1U illustrate cross-sections taken through portions of hub 160 as indicated in FIGS. 1M and 1R. Receptacle(s) 166 can include an interior 166i that is sized and/or shaped to receive at least a portion of arms 174b of catheter hub holder 174. In some implementations, frame 164 includes one receptacle 166 and catheter hub holder 174 includes one arm 174. In some implementations, frame 164 includes two receptacles 166 and catheter hub holder 174 includes two arms 174b, such as is shown in the figures. In some implementations in which frame 164 includes two receptacles 166, such two receptacles 166 are substantially aligned with another (for example, along an axis extending through a center of interiors 166i of receptacles 166). In some implementations, arms 174b are substantially aligned with another. Body 174a of catheter hub holder 174 can be configured to secure to catheter hub 142. In some implementations, body 174a comprises a C-shape. In some implementations, body 174a is configured to surround at least about 25%, at least about 50%, or at least about 75% of a cross-section of catheter hub 142. In some implementations, body 174a is shaped to secure around a portion of a cylindrical section of catheter hub 142. In some implementations, body 174a is shaped to surround less than an entire perimeter of a cross-section of catheter hub 142 (for example, a circular cross-section of catheter hub 142). In some implementations, body 174a is configured so as to not allow relative movement between body 174a and catheter hub 142 when secured together. In some implementations, body 174a is configured to inhibit inadvertent disconnection between catheter hub holder 174 and catheter hub 142.

Catheter hub holder 174 can be movably coupled to frame 164 and/or membrane 162, for example, via arm(s) 174b and receptacle(s) 166. FIGS. 1S-1U illustrate three reference axes 101, 103, and 105 that are perpendicular to one another. With reference to FIG. 1R-1U, a width W1 of hub 160 extends along axis 105 and a length L₁ of hub 160 extends along axis 103. A height of hub 160 (and/or catheter stabilization device 100) extends along axis 101. FIGS. 1T-1U illustrates cross-sectional views through receptacles 166 and catheter hub holder 174. As shown, interiors 166i of receptacles 166 can have a height 166h and width 166w. As also shown, arms 174b can have a height 174h (which also may be referred to as a thickness of arms 174b) and a width 174w. Height 166h and height 174h extend along axis 101. Width 166w and width 174w extend along axis 105. In some implementations, height 174h is less than height 166h. Such implementations allow arms 174b (and catheter hub holder 174) to move in one or both directions along axis 101 within interior 166i (see FIG. 1T). In some implementations, width 174w is less than width 166w. Such implementations allow arms 174b (and catheter hub holder 174) to move in one or both directions along axis 105 within interior 166i (see FIG. 1U). In some implementations, a distance 174d between free ends of arms 174b is less than a distance 166d between outer portions of interior 166i of receptacles 166 (see FIG. 1T). Such implementations allow arms 174b (and catheter hub holder 174) to move in one or both directions along axis 103.

In some implementations, arms 174b and receptacles 166 are configured to allow relative movement (between catheter hub holder 174 and frame 164/membrane 162) with respect to all of such axes 101, 103, 105. In some variants, arms 174b and receptacles 166 are configured to allow relative movement (between catheter hub holder 174 and frame 164/membrane 162) only with respect to two of such axes 101, 103, 105. For example, in some variants: height 174h is less than height 166h such that movement of arms 174b within interior 166i along axis 101 is permitted; width 174w is less than width 166w such that movement of arms 174b within interior 166i along axis 105 is permitted; and distance 174d is substantially equal to distance 166d such that movement of arms 174b within interior 166i along axis 103 is inhibited. In some variants: width 174w is less than width 166w such that movement of arms 174b within interior 166i along axis 105 is permitted; height 174h is substantially equal to height 166h such that movement of arms 174b within interior 166i along axis 101 is inhibited; and distance 174d is less than distance 166d such that movement of arms 174b within interior 166i along axis 103 is permitted. In some variants: distance 174d is less than distance 166d such that movement of arms 174b within interior 166i along axis 103 is permitted; height 174h is less than height 166h such that movement of arms 174b within interior 166i along axis 101 is permitted; and width 174w is substantially equal to width 166w such that movement of arms 174b within interior 166i along axis 105 is inhibited.

In some variants, arms 174b and receptacles 166 are configured to allow relative movement (between catheter hub holder 174 and frame 164/membrane 162) only with respect to one of such axes 101, 103, 105. For example, in some variants, arms 174b and receptacles 166 are configured to allow relative movement: only along axis 101; only along axis 103; or only along axis 105. In some variants: height 174h is less than height 166h such that movement of arms 174b within interior 166i along axis 101 is permitted; width 174w is substantially equal to width 166w such that movement of arms 174b within interior 166i along axis 105 is inhibited; and distance 174d is substantially equal to distance 166d such that movement of arms 174b within interior 166i along axis 103 is inhibited. In some variants: width 174w is less than width 166w such that movement of arms 174b within interior 166i along axis 105 is permitted; height 174h is substantially equal to height 166h such that movement of arms 174b within interior 166i along axis 101 is inhibited; and distance 174d is substantially equal to distance 166d such that movement of arms 174b within interior 166i along axis 103 is inhibited. In some variants: distance 174d is less than distance 166d such that movement of arms 174b within interior 166i along axis 103 is permitted; height 174h is substantially equal to height 166h such that movement of arms 174b within interior 166i along axis 101 is inhibited; and width 174w is substantially equal to width 166w such that movement of arms 174b within interior 166i along axis 105 is inhibited.

In some implementations, catheter hub holder 174, frame 164, and/or membrane 162 are configured to allow relative rotation. For example, in some implementations, catheter hub holder 174 can be configured to rotate relative to frame 164 and/or membrane 162. As an example, arms 174b (which can be connected to body 174a) and receptacles 166 can be rotatably coupled to one another.

With reference to FIG. 1U, in some implementations, rotation of arms 174b within interiors 166i, for example, about an axis extending through arm 174b and/or interior 166i (for example, parallel to axis 103) is permitted. In some implementations, arm 174b can rotate within interior 166i about an axis extending through arm 174 and/or interior 166i an amount that is between about 0 degrees and about 60 degrees, between about 0 degrees and about 55 degrees, between about 0 degrees and about 50 degrees, between about 0 degrees and about 45 degrees, between about 0 degrees and about 40 degrees, between about 0 degrees and about 35 degrees, between about 0 degrees and about 30 degrees, between about 0 degrees and about 25 degrees, between about 0 degrees and about 20 degrees, between about 0 degrees and about 15 degrees, between about 0 degrees and about 10 degrees, or between about 0 degrees and about 5 degrees. In some implementations, rotation of arms 174b within interiors 166i, for example, about an axis extending through arm 174b and/or interior 166i is limited to an amount that is less than or equal to about 60 degrees, less than or equal to about 55 degrees, less than or equal to about 50 degrees, less than or equal to about 45 degrees, less than or equal to about 40 degrees, less than or equal to about 35 degrees, less than or equal to about 30 degrees, less than or equal to about 25 degrees, less than or equal to about 20 degrees, less than or equal to about 15 degrees, less than or equal to about 10 degrees, or less than or equal to about 5 degrees. Arms 174b can be sized and/or shaped relative to a size and/or shape of interior 166i of receptacles 166 such that rotation of arms 174b within interiors 166i is limited to the above-described amounts. In some implementations, arms 174b have a non-circular shape (such as oblong) and/or the interiors 166i have a non-circular shape (such as oblong).

With reference to FIG. 1T, in some implementations, rotation of arms 174b within interiors 166i relative to axis 105 (and/or an axis extending through a center of a cross-section of catheter hub holder 174) is permitted. In some implementations, arm 174b can rotate within interior 166i relative to axis 105 (and/or an axis extending through a center of a cross-section of catheter hub holder 174) an amount that is between about 0 degrees and about 60 degrees, between about 0 degrees and about 55 degrees, between about 0 degrees and about 50 degrees, between about 0 degrees and about 45 degrees, between about 0 degrees and about 40 degrees, between about 0 degrees and about 35 degrees, between about 0 degrees and about 30 degrees, between about 0 degrees and about 25 degrees, between about 0 degrees and about 20 degrees, between about 0 degrees and about 15 degrees, between about 0 degrees and about 10 degrees, or between about 0 degrees and about 5 degrees. In some implementations, rotation of arms 174b within interiors 166i relative to axis 105 (and/or an axis extending through a center of a cross-section of catheter hub holder 174) is limited to an amount that is less than or equal to about 60 degrees, less than or equal to about 55 degrees, less than or equal to about 50 degrees, less than or equal to about 45 degrees, less than or equal to about 40 degrees, less than or equal to about 35 degrees, less than or equal to about 30 degrees, less than or equal to about 25 degrees, less than or equal to about 20 degrees, less than or equal to about 15 degrees, less than or equal to about 10 degrees, or less than or equal to about 5 degrees.

With continued reference to FIG. 1T, in some implementations, rotation of arms 174b within interiors 166i relative to axis 101 is permitted. In some implementations, arms 174b can rotate within interiors 166i relative to axis 101 an amount that is between about 0 degrees and about 60 degrees, between about 0 degrees and about 55 degrees, between about 0 degrees and about 50 degrees, between about 0 degrees and about 45 degrees, between about 0 degrees and about 40 degrees, between about 0 degrees and about 35 degrees, between about 0 degrees and about 30 degrees, between about 0 degrees and about 25 degrees, between about 0 degrees and about 20 degrees, between about 0 degrees and about 15 degrees, between about 0 degrees and about 10 degrees, or between about 0 degrees and about 5 degrees. In some implementations, rotation of arms 174b within interiors 166i relative to axis 401 is limited to an amount that is less than or equal to about 60 degrees, less than or equal to about 55 degrees, less than or equal to about 50 degrees, less than or equal to about 45 degrees, less than or equal to about 40 degrees, less than or equal to about 35 degrees, less than or equal to about 30 degrees, less than or equal to about 25 degrees, less than or equal to about 20 degrees, less than or equal to about 15 degrees, less than or equal to about 10 degrees, or less than or equal to about 5 degrees.

In some implementations: rotation of arms 174b within interiors 166i relative to axis 101 and/or 103 is permitted (such as to any of the above-mentioned values or ranges); and rotation of arms 174b within interiors 166i relative to axis 105 is limited to an amount that is less than or equal to any of the above-mentioned values. In some implementations: rotation of arms 174b within interiors 166i relative to axis 101 and/or 105 is permitted (such as to any of the above-mentioned values or ranges); and rotation of arms 174b within interiors 166i relative to axis 103 is limited to an amount that is less than or equal to any of the above-mentioned values. In some implementations: rotation of arms 174b within interiors 166i relative to axis 103 and/or 105 is permitted (such as to any of the above-mentioned values or ranges); and rotation of arms 174b within interiors 166i relative to axis 101 is limited to an amount that is less than or equal to any of the above-mentioned values. In some implementations, rotation of arms 174b within interiors 166i is only permitted relative to one of axes 101, 103, or and 105. For example, in some implementations, rotation of arms 174b within interiors 166i is only permitted relative to axis 103. Permittance of rotation relative to axis 102 can allow catheter hub 142 and catheter 141 to have self-adjustable inclination relative to the skin surface and/or catheter insertion site.

FIGS. 1X and 1Y illustrate additional implementations of catheter hub holders 174′, 174″, which can include body 174a′, 174a″ and arms 174b′, 174b″. Body 174a′, 174a″ can have a different size and/or shape than body 174a, for example, to allow securement to catheter hubs having different sizes and/or shapes. Arms 174b′, 174b″ can have a different length to accommodate the different sized/shaped bodies 174a′, 174a″ and to allow catheter hub holders 174′, 174″ to be utilized with receptacles 166. Body 174a can be smaller than body 174a′ and larger than body 174a″. Arms 174b can be smaller than arms 174b″ and larger than arms 174b′. A kit (which may also be referred to as an “assembly”) can include frame 164 and/or membrane 162 and a plurality of catheter hub holders (such as two or more of catheter hub holders 174, 174′, 174″) having different sizes and/or shapes corresponding to different sizes and/or shapes of catheter hubs, wherein the frame 164 and/or membrane 162 (for example, receptacles 166) are configured to be removably coupled (for example, movably and/or rotatably coupled as described herein) to each of said plurality of catheter hub holders.

As discussed above, catheter stabilization device 100 can include a first portion configured to secure to the subject's skin and a second portion configured to secure to catheter hub 142 coupled to catheter 141 that is configured for insertion at a catheter insertion site. As also discussed above, frame 164 and/or membrane 162 can be an implementation of such “first portion” and catheter hub holder 174 can be an implementation of such “second portion”. Although frame 164 is described above as having receptacles 166 and catheter hub holder 174 is described above as having arms 174b, in some variants, frame 164 has arm(s) (that can be similar or identical to arms 174b) and catheter hub holder 174 has receptacle(s) (that can be similar or identical to receptacles 166). FIGS. 1AA-1BB illustrates an implementation of such variants. As shown, frame 164′ (which can be similar to frame 164) includes arms 174b′ extending from a portion of a frame body 170′ (for example, towards one another). Frame body 170′ can be similar or identical to frame body 170. FIGS. 1AA-1BB also illustrate an alternative implementation of a catheter hub holder 174′″ that includes a body 174a′″ (which can be similar to body 174a) and receptacles 166′ which extend from body 174a′″ (for example, in opposite directions). As shown, arms 174b′ of frame 164′ can be positioned at least partially within interiors 166i′ of receptacles 166′. The movement and/or rotation of arms 174b′″ and receptacles 166′ can be similar or identical to that which is described and/or shown herein with respect to arms 174b and receptacles 166. The dimensions and/or other characteristics (for example, shape) of arms 174b′″ can be similar or identical to the dimensions and/or characteristics of arms 174b described above, and the dimensions and/or other characteristics (for example, shape) of receptacles 166′ (for example, interiors 166i′) can be similar or identical to the dimensions and/or characteristics of receptacles 166 described above (for example, interiors 166).

As mentioned previously, the present disclosure provides devices having a first portion securable to the subject's skin and a second portion that can stabilize (for example, operably position) a catheter inserted at a catheter insertion site. As also described herein, such first and second portions can be movably coupled to one another to allow for relative movement between the first portion (which secures to the subject's skin) and the catheter hub and connected catheter. Allowing relative movement between such first portion and the catheter hub and connected catheter can in turn inhibit relative movement between the catheter and the vein and/or catheter insertion site, which can be desirable for reasons described above. While various devices disclosed herein have been described with reference to a catheter and a catheter hub, the present disclosure extends beyond this particular context. The present disclosure contemplates devices that can secure other types of components (such as medical components) to a subject and can allow for relative movement between the secured component and a portion of the subject's body to which the device is secured. For example, the present disclosure contemplates a device having a first portion that can be secured to skin on a portion of a subject's body (for example, at or near any of a variety of open wound sites on a subject's body) and a second portion that is movably coupled with such first portion and that is configured to secure to a component such as a tube, wire (such as an ECG wire), a blood glucose measurement device, a catheter intended for insertion in a vein or through the skin into another region of a subject's body (such as kidneys), among other components. Such component to which the second portion secures to can be a partially implantable device. Such “first” and “second” portions of the device can be similar to any of the implementations described herein, for example, with respect to catheter hub holder 174 and receptacles 166.

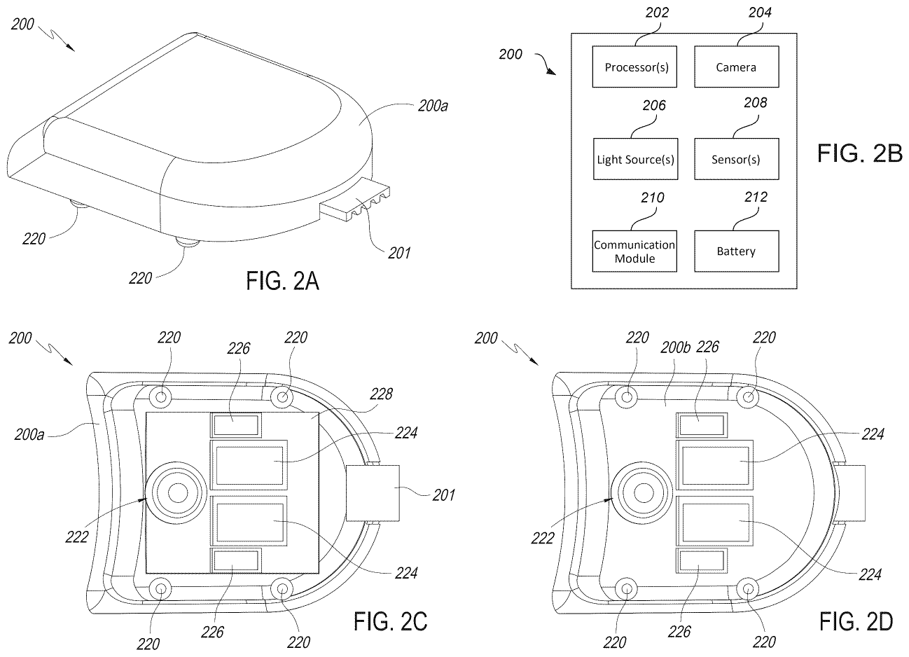

FIG. 2A illustrates a top perspective view of an electronic device 200. Electronic device 200 can be configured to connect to (for example, removably connect to) any of the catheter stabilization devices disclosed herein. Electronic device 200 can include various electronic components, such as those described further below. FIG. 2D illustrates a bottom view of electronic device 200. Electronic device 200 can include a housing comprising a top shell 200a and a bottom shell 200b, which can at least partially enclose and/or protect any of the electronic components contained within such housing. Such housing can be waterproof, which can advantageously allow electronic device 200 to be wiped with and/or submerged in disinfectant without allowing such disinfectant to reach electronic components of electronic device 200. FIG. 2C illustrates a bottom view of electronic device 200 with bottom shell 200b removed.

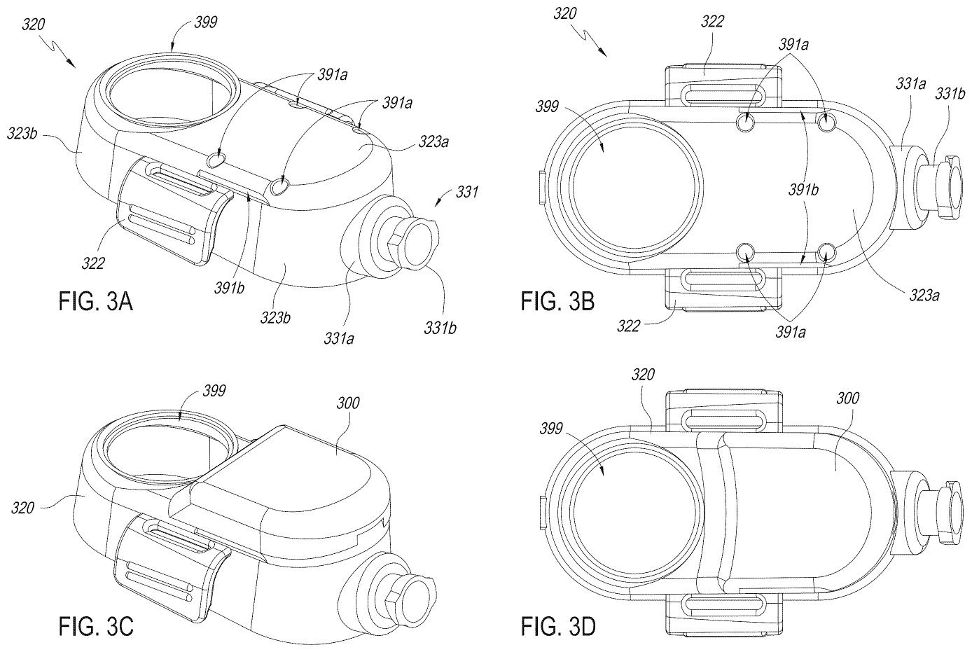

FIGS. 3A-3B illustrate top perspective and top views (respectively) of another implementation of a cover 320. Cover 320 can be similar or identical to any of the covers disclosed herein in some or many respects. Cover 320 can include a top portion 323a, wall 323b, wings 322, and/or a connector 331 comprising a connector body 331a and threaded portion 331b. Top portion 323a, wall 323b, wings 322, connector 331, connector body 331a, and threaded portion 331b can be similar or identical to top portion 123a, wall 123b, wings 122, connector 131, connector body 131a, and threaded portion 131b of cover 120. Cover 320 can be configured to removably connect to electronic device 200. For example, in some implementations, cover 320 includes one or more (such as one, two, three, four, five, or six) cavities 391a that are configured to receive one or more (such as one, two, three, four, five, or six) pins 220 of electronic device 200 (see FIG. 2C-2D). The engagement between pin(s) 220 and cavities 391a can be, for example, a press-fit engagement. FIGS. 3C-3D illustrate how electronic device 200 and cover 320 can be connected together via pins 220 and cavities 391a. In some variants, electronic device 200 includes one or more cavities (which can be similar or identical to cavities 391a) and cover 320 includes one or more pins (which can be similar or identical to pins 220) that can secure to one another.

In addition or as an alternative to pins 220 and cavities 391a, electronic device 200 and cover 320 can be configured to slidably connect to one another. For example, in some implementations, cover 320 includes one or more grooves 391b (such as one or two grooves 391b) and electronic device 200 includes one or more protrusions (for example, along a perimeter of electronic device 200 or a housing thereof) that can slidably engage such grooves 391b. Such grooves 391b can be located at or near opposite portions of wall 323b and/or near a location where top portion 323a joins wall 323b. Such grooves 391b can comprise a tapered profile. In some variants, electronic device 200 include one or more grooves (which can be similar or identical to grooves 391b) and cover 320 includes one or more protrusions that can slidably engage such grooves. Although such cavities 391a and grooves 391b are described with respect to cover 320, any of the other covers disclosed herein can incorporate such cavities 391a and/or grooves 391b in order to connect to electronic device 200.

FIG. 2B illustrates an example schematic diagram of electronic device 200. Electronic device 200 can include a processor (or one or more processors) 202 and a communication module 210. In some implementations, electronic device 200 includes a battery 212. In some variants, electronic device 200 does not include a battery, but receives power from an external power source (for example, via a cable). In some implementations, electronic device 200 includes a camera 204, one or more light sources 206, and/or one or more sensors 208 (such as physiological measurement sensors). In some implementations in which electronic device 200 includes battery 212, electronic device 200 is configured to allow wireless charging of battery 212 (for example, inductive charging). In some implementations, electronic device 200 includes a connector port configured to electrically connect to a connector port of an external device (for example, for charging and/or communication). FIGS. 2A and 2C illustrate an example electrical component 201 that can be associated with an external device (or connected to an external device) in a position within a portion of housing (for example, shell 200a). FIG. 2D illustrates electronic device 200 without also showing electrical component 201. In some implementations, electronic device 200 is configured for both of wireless and wired communication with external device(s). Such implementations can provide a form of redundancy for communication if one of such communication methods is impaired.

Processor 202 can be configured, among other things, to process data, execute instructions to perform one or more functions, and/or control operation of electronic device 200. For example, processor 202 can process data obtained from electronic device 200 (for example, from camera 204 and/or sensor(s) 208) and can execute instructions to perform functions related to storing and/or transmitting such data.

Communication module 210 can facilitate communication (via wired and/or wireless connection) between electronic device 200 (and/or components thereof) and separate external devices, such as separate monitoring and/or mobile devices. For example, communication module 210 can be configured to allow electronic device 200 to wirelessly communicate with other devices, systems, and/or networks over any of a variety of communication protocols. Communication module 210 can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. Communication module 210 can allow data and/or instructions to be transmitted and/or received to and/or from electronic device 200 and separate external computing devices. Communication module 210 can be configured to transmit (for example, wirelessly) processed and/or unprocessed data (for example, from camera 204 and/or sensor(s) 208) or other information to separate computing external devices, which can include, among others, a mobile device (for example, an iOS or Android enabled smartphone, tablet, laptop), a desktop computer, a server or other computing or processing device for display and/or further processing, among other things. Such separate computing devices can be configured to store and/or further process the received data and/or other information, to display the received data and/or information indicative of or derived from the received data, and/or to transmit the data (including displays, alarms, alerts, and notifications) to various other types of computing devices and/or systems that may be associated with a hospital, a caregiver (for example, a primary care provider), and/or a user that have permission to access the subject's data. As another example, communication module 210 of electronic device 200 can be configured to wirelessly transmit processed and/or unprocessed data to a separate computing device which can include one or more processors configured to execute an application that generates a graphical user interface displaying the data and/or other information obtained from electronic device 200.

Electronic device 200 can be configured to connect to (for example, removably connect to) any of the catheter stabilization devices disclosed herein. As discussed herein, electronic device 200 can be configured to connect to cover 320. Camera 204 can be configured to generate visual signals (for example, images) of a catheter insertion site, a catheter inserted at the catheter insertion site, a catheter hub, and/or any other regions near any of these and/or enclosed by cover 320 to which electronic device 200 is secured. In some implementations, processor 202 is configured to receive such visual signals from camera 204 and determine a condition (which may also be referred to as a “status”) of the catheter insertion site, the catheter, and/or the catheter hub. Such determination can involve comparing visual signal(s) generated by camera 204 at a first time period with visual signal(s) generated by camera 204 at a second time period. Additionally or alternatively, such determination can involve comparing visual signal(s) generated by camera 204 at one or more time periods to one or more visual signal(s) (for example, images) in a database. Such database can store, for example, a plurality of images of catheter insertion sites and/or relative positioning of catheters and/or catheter hubs and can be utilized as a reference point. In some implementations, visual signals generated by camera 204 are transmitted (for example, wirelessly) to an external device for analysis, for example, via communication module 210. One or more processors on such external device can be configured to operate in a similar or identical manner as that described above with respect to processor 202. Whether the determination of the condition of the catheter insertion site, the catheter, and/or the catheter hub is made by the electronic device 200, or an external device in communication with electronic device 200, visual signal(s) generated by camera 204 can be utilized not only for real time visualization/visibility by a caregiver (or another), but also can be utilized to diagnose (in an automated manner) an infection in its early stages and/or can be utilized to assess whether attention should be given to the catheter, catheter insertion site, and/or catheter hub (for example, replacing and/or repositioning any of the same).

In some implementations, electronic device 200 includes one or more light sources 206 configured to emit light of one or more wavelengths at and/or toward: a catheter insertion site; a catheter inserted at the catheter insertion site; a catheter hub; and/or any other regions near any of these and/or enclosed by a cover to which electronic device 200 is secured. Such light source(s) 206 can include: one or more wide spectrum LEDs configured to emit light having wavelengths between a first range; one or more narrow spectrum LEDs configured to emit light having wavelengths between a second range that is less than said first range; and/or one or more UV LEDs. Such first range can be, for example, between about 380 nm and about 740 nm. Such UV LEDs can include: one or more UVA LEDs; one or more UVB LEDs; and/or one or more UVC LEDs. In some implementations, the one or more UVA LEDs are configured to emit light at wavelengths between about 320 nm and about 400 nm; the one or more UVB LEDs are configured to emit light at wavelengths between about 290 nm and about 320 nm; and the one or more UVC LEDs are configured to emit light at wavelengths between about 200 nm and about 280 nm. Inclusion of one or more UV LEDs, such as any of the UVA, UVB, and/or UVC LEDs described above can provide for disinfection of the catheter insertion site, catheter, catheter hub, and/or other regions nearby.

Inclusion of any of the above-described wide spectrum and/or narrow spectrum LEDs can be utilized for determining physiological parameters such as blood oxygen saturation and/or pulse rate, for example, where electronic device 200 include one or more detectors (which can be part of sensor(s) 208) configured to detect the emitted light after attenuating through skin tissue. Additionally or alternatively, such LEDs can be utilized to illuminate varying colors which can be utilized to gather additional visual data points with imagery obtained using camera 204. For example, visual data can be obtained using camera 204 across one or more time periods where such LEDs provide illumination with varying colors, and the imagery obtained using different colors can allow for additional analysis of the condition of the catheter insertion site, catheter, catheter hub, and/or other regions nearby.

Sensor(s) 208 can include any of a variety of sensors for measuring one or more physiological parameters of a subject. For example, sensor(s) 208 can include: a temperature sensor (for example, for determining skin temperature and/or temperature within a region enclosed by any of the catheter stabilization devices to which electronic device 200 is secured); a humidity sensor; and/or a pulse oximetry sensor for determining pulse rate and/or blood oxygen saturation.

With reference to FIGS. 2C-2D, electronic device 200 can include a circuit board 228, a camera 222 (which can be an implementation of camera 204), wide and/or narrow spectrum LEDs 226 (which can be implementations of those discussed previously with respect to light sources 206), and/or UV LEDs 224 (which can be implementations of those discussed previously with respect to light sources 206). Camera 222, LEDs 224, and/or LEDs 226 can be connected to a surface of circuit board 228 so as to face toward the catheter insertion site, catheter, catheter hub, and/or skin enclosed and/or surrounded by a cover to which electronic device 200 is secured during use. In some implementations, camera 222 comprises a wide-angle lens.

Cover 320 can be configured to at least partially enclose a catheter insertion site on a subject. With reference to FIGS. 3A-3D, cover 320 can include an opening 399 configured to provide visibility of the catheter insertion site (and/or the inserted catheter or connected catheter hub) when cover 320 is in use. In some implementations, a transparent material is placed over opening 399, which can seal the enclosed region. In some implementations, cover 320 includes a magnifying lens positioned within and/or over opening 399. Such magnifying lens can advantageously magnify a view of a catheter insertion site (and/or the inserted catheter or connected catheter hub), for example, to two, three, or four times its actual size. Although such opening 399 and magnifying lens are described with respect to cover 320, any of the other covers disclosed herein can incorporate such opening 399 and magnifying lens. In some implementations, cover 320 is made of a transparent material, which can allow camera 204 to inspect, and/or the light source(s) 206 to emit light at, the catheter insertion site, catheter, catheter hub, and/or skin enclosed and/or surrounded by cover 320 when electronic device 200 is secured to cover 320 (for example, top portion 323a of cover 320). With reference to FIGS. 3C-3D, in some implementations, electronic device 200 connects to a portion of cover 320 that is spaced from opening 399 so as to not interfere with viewing of the catheter insertion site, catheter, and/or catheter hub via opening 399 or a magnifying lens over opening 399.

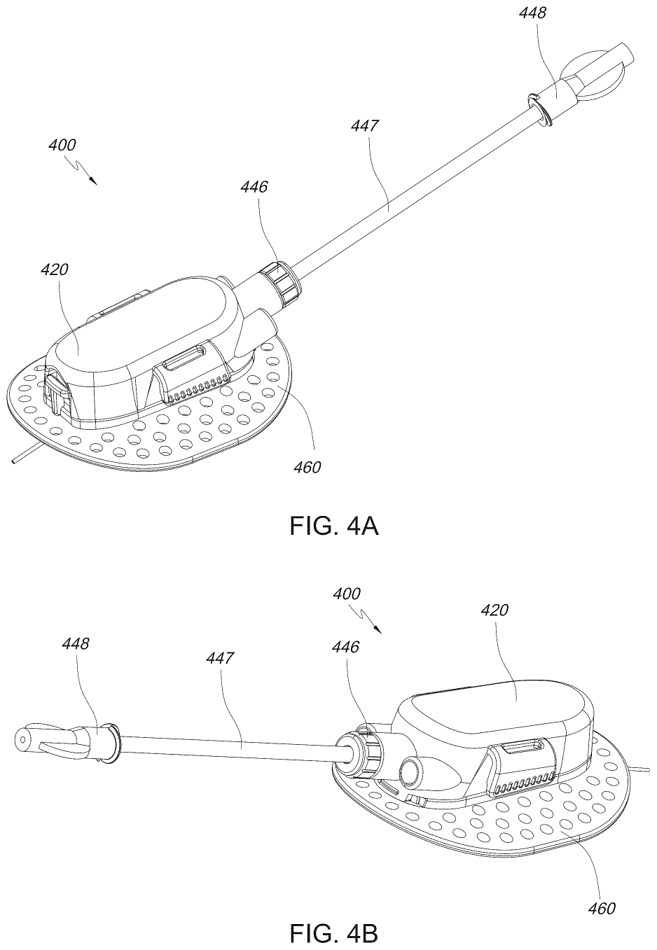

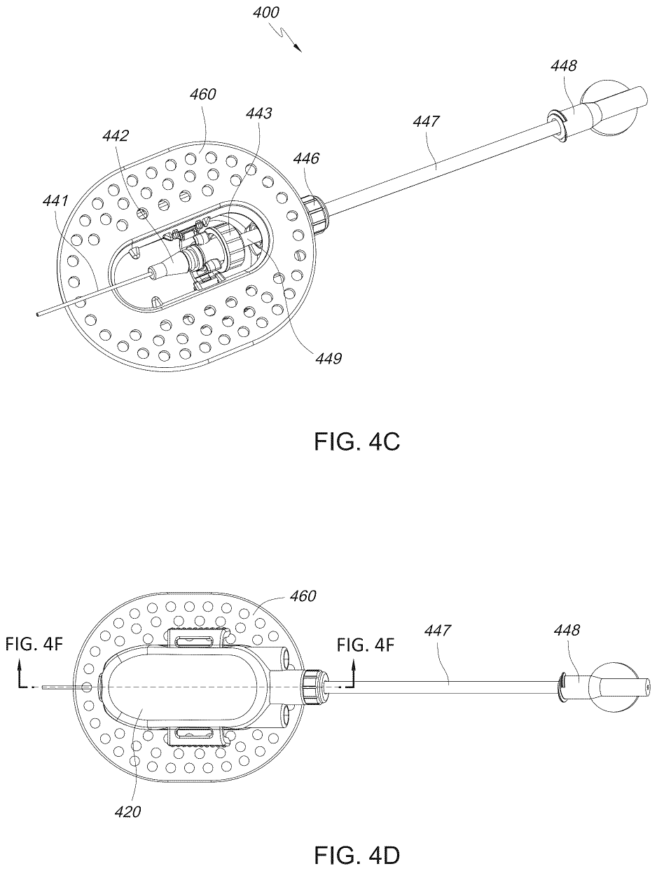

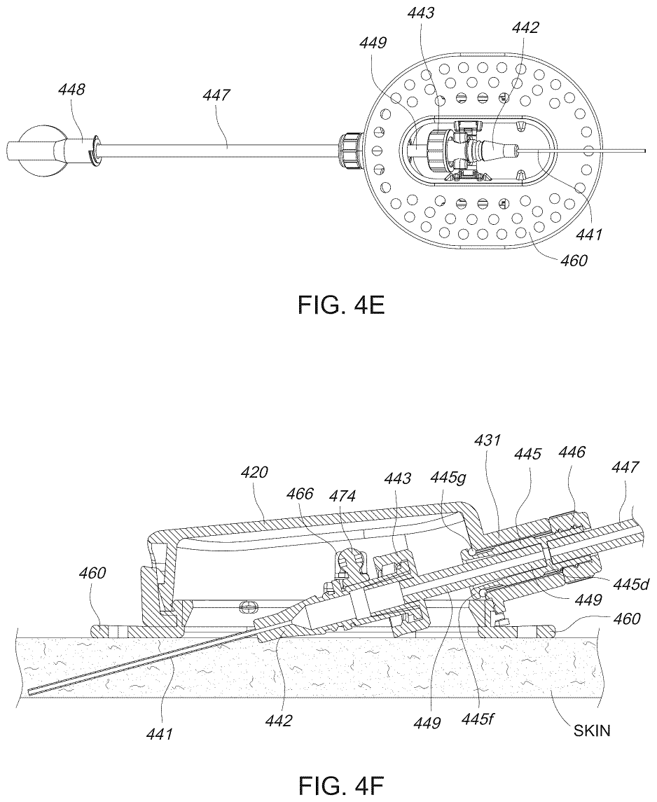

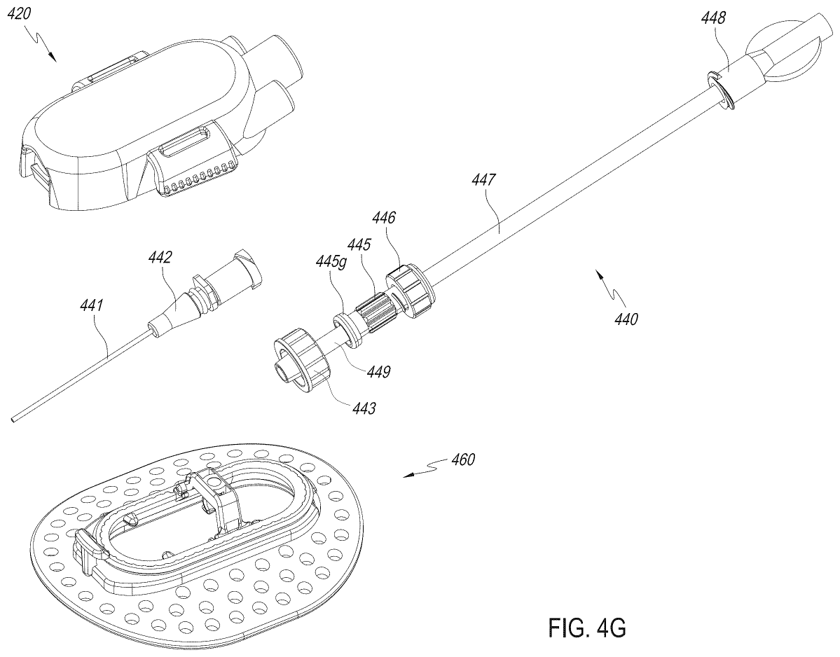

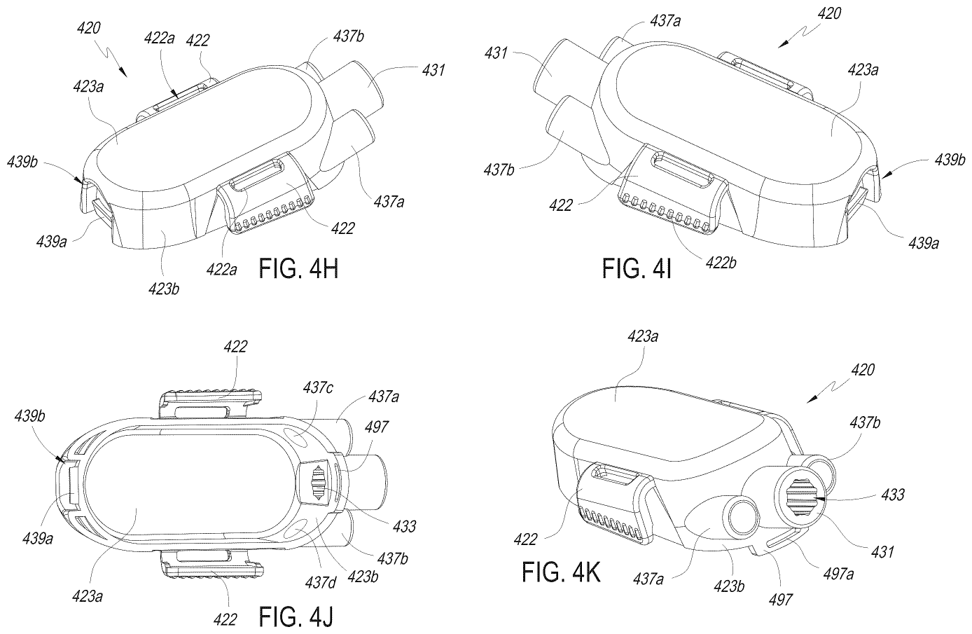

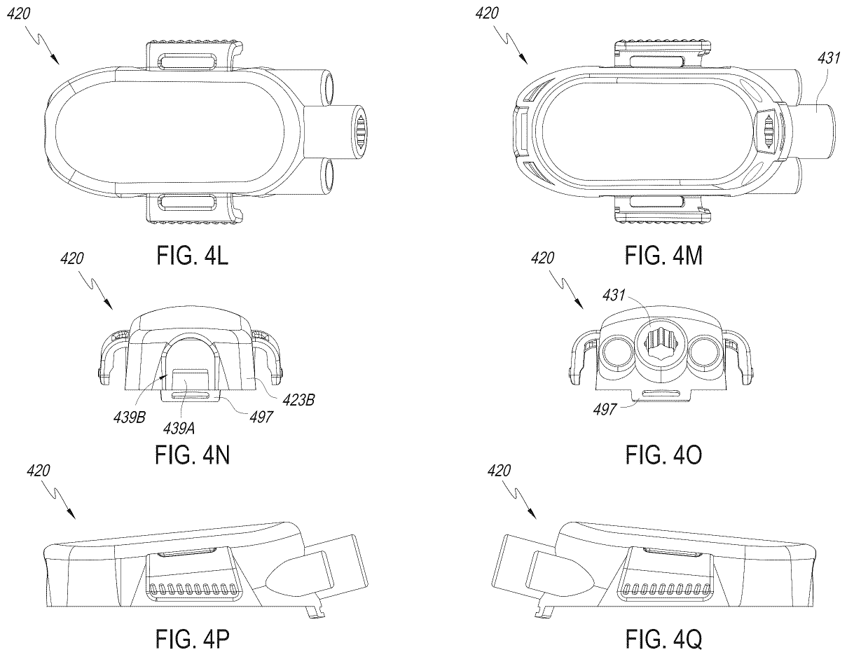

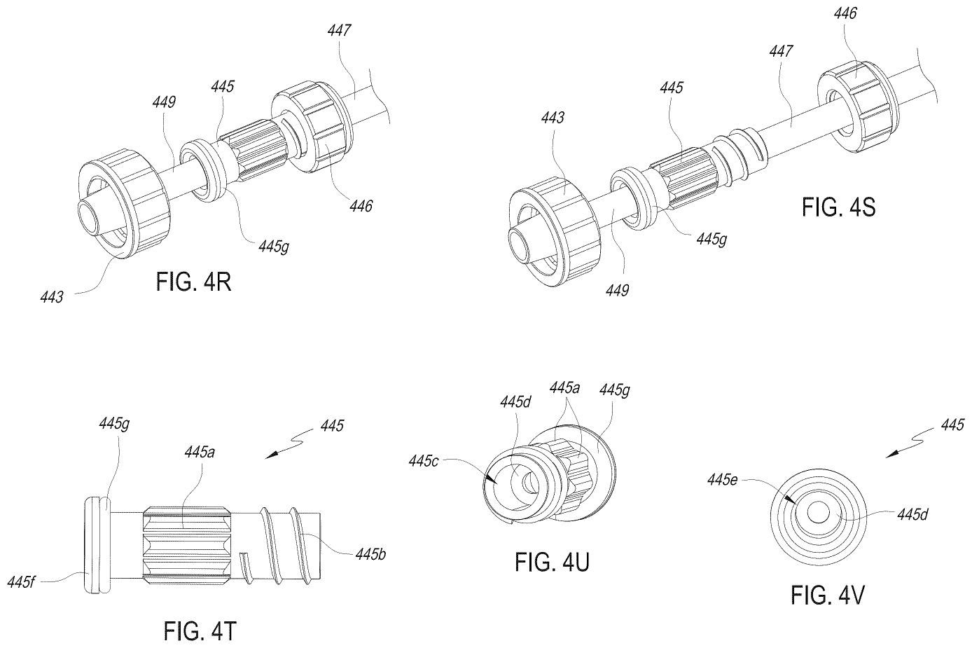

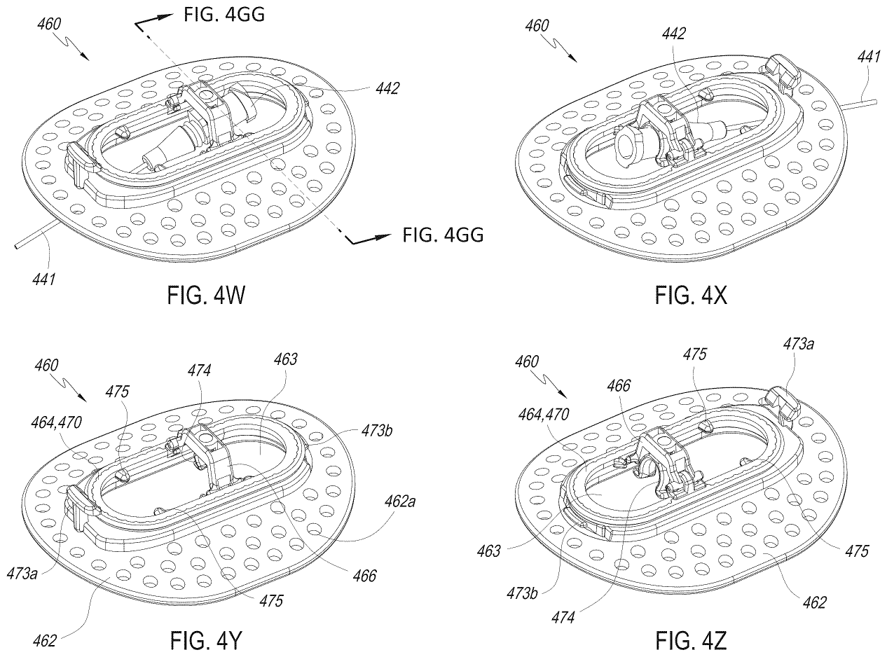

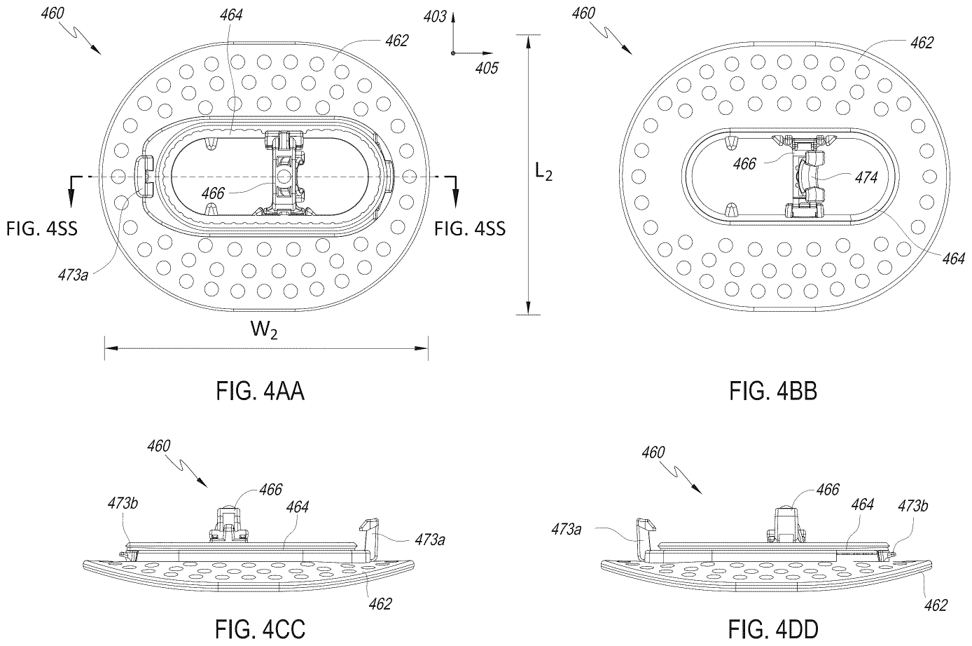

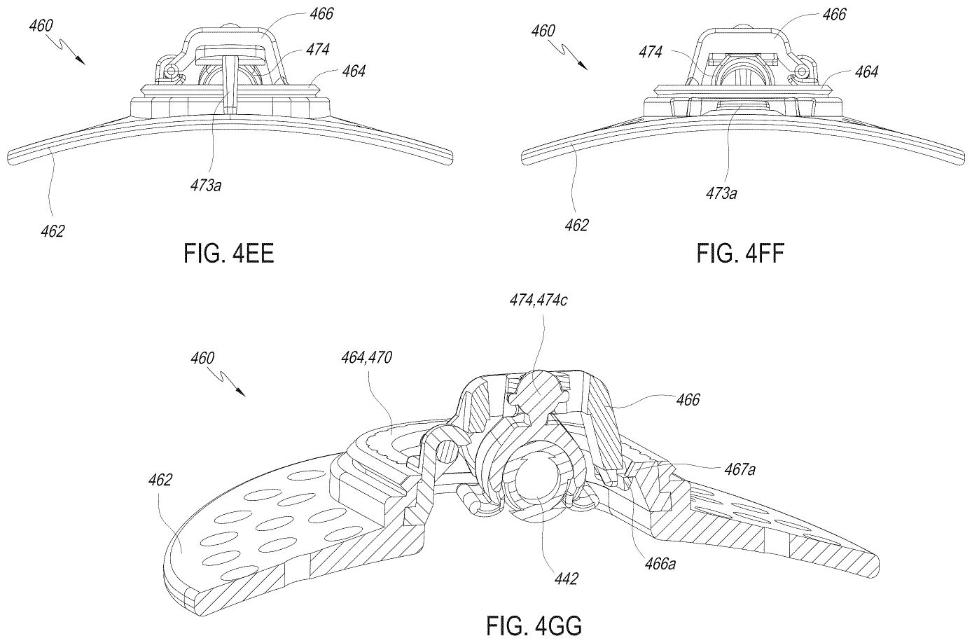

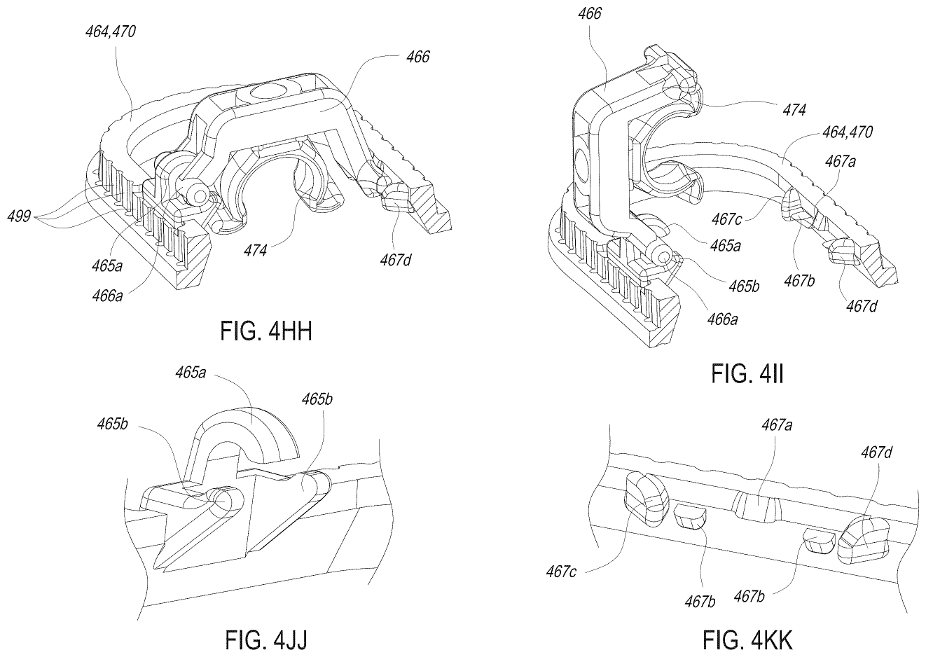

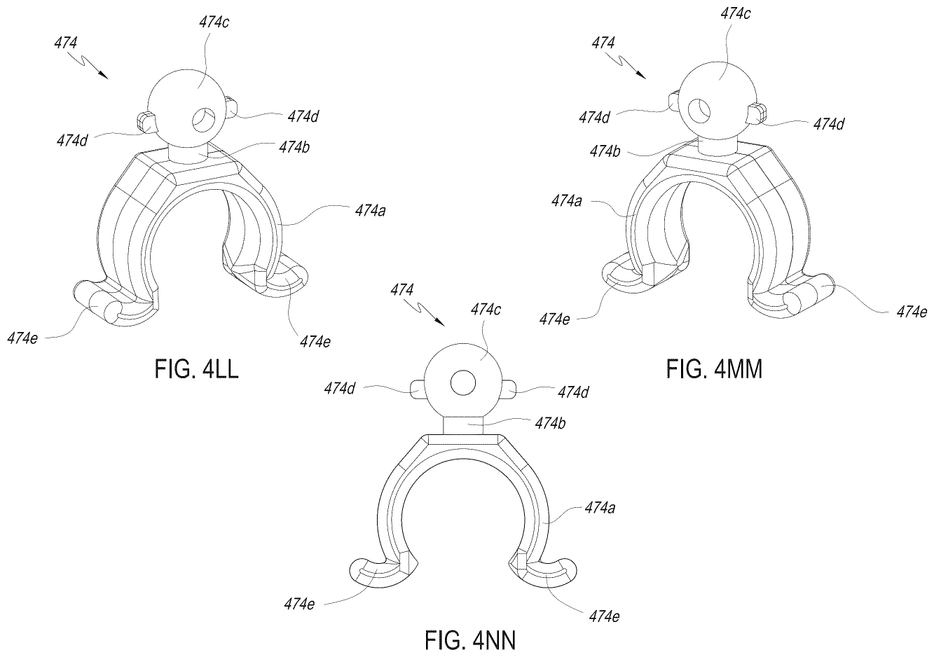

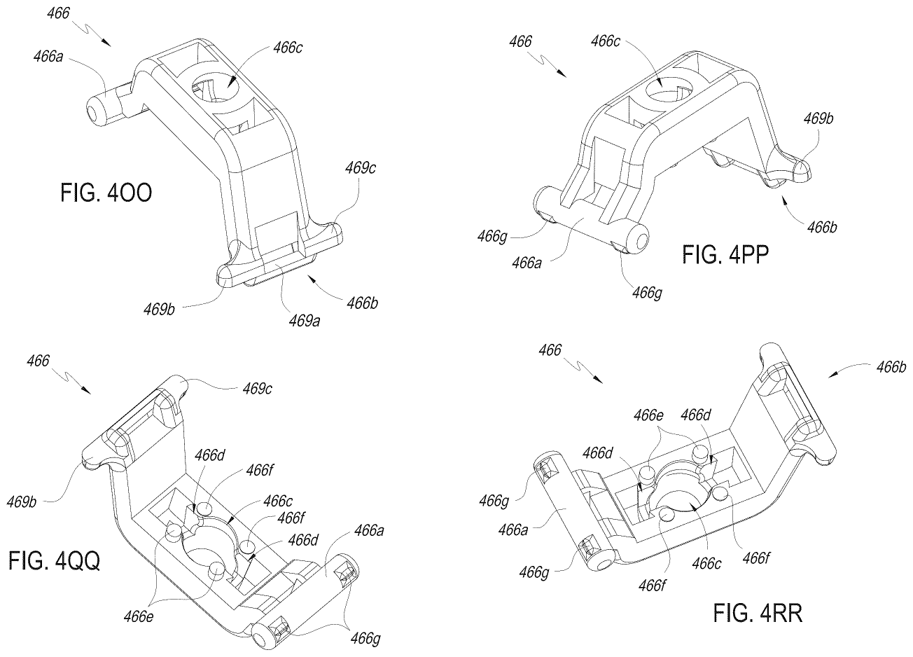

FIGS. 4A-4G illustrate another implementation of a catheter stabilization device 400 and various portions thereof. Catheter stabilization device 400 (which may also be referred to as a “catheter housing”, “catheter housing device”, “catheter securement device”, “catheter stabilization system”, or “device”) can be similar in some or many respects to catheter stabilization device 100. FIGS. 4A-4B illustrate top perspective views of, and FIG. 4C illustrates a bottom perspective view of, catheter stabilization device 400. FIGS. 4D and 4E illustrate top and bottom views (respectively) of catheter stabilization device 400. FIG. 4F illustrates a cross-section taken through a portion of catheter stabilization device 400 (as indicated in FIG. 4D) in a condition of use where device 400 is secured to skin of a subject. Although not illustrated in FIG. 4F, it is to be understood that catheter 441 can be at least partially positioned within a vein of the subject when in use. FIG. 4G illustrates an exploded view of catheter stabilization device 400 proximate a catheter 441 and a catheter hub 442. Catheter 441 can be similar or identical to catheter 141 and catheter hub 442 can be similar or identical to catheter hub 142. With reference to FIG. 4G, catheter stabilization device 400 can include a cover 420 and a hub 460. In some implementations, catheter stabilization device 400 includes an extension set 440. In some implementations, cover 420 and extension set 440 are coupled to one another, for example, in a similar manner as described with respect to any of the covers and extension sets in PCT. Pub. No. WO2022/140633A1. In some variants, catheter stabilization device 400 includes hub 460 (or hub 460′ or hub 460″, each described further below) but does not include cover 420 and/or does not include extension set 440. In some variants, catheter stabilization device 400 includes cover 420 but does not include hub 160 and/or does not include extension set 440.

FIGS. 4H-4Q illustrate views of cover 420. FIGS. 4H-4I and 4K illustrate top perspective views of cover 420 and FIG. 4J illustrates a bottom perspective view of cover 420. FIGS. 4L and 4M illustrate top and bottom views of cover 420 (respectively). FIGS. 4N and 40 illustrate front and rear views of cover 420 (respectively). FIGS. 4P and 4Q illustrate side views of cover 420. Cover 420 can be similar to cover 120 in some or many respects. Cover 420 can include a top portion 423a, a wall 423b, and/or one or more wings 422 that can be similar to top portion 123a, wall 123b, and/or wings 122 (respectively) of cover 120 described above in some of many respects. In some implementations, wings 122 include an opening 122a (which can be similar or identical to opening 122a of wing 122). In some implementations, wings 122 include one or a plurality of protrusions 422b that can aid a user in gripping cover 420. Cover 420 can include one or both of ports 437a, 437b that can extend from a portion of cover 420, such as wall 423b (or alternatively, from top portion 423a). Ports 437a, 437b can be similar or identical to ports 137a, 137b or any of the ports disclosed in PCT. Pub. No. WO2022/140633A1. With reference to FIG. 4J, cover 420 can include openings 437c, 437d to facilitate fluid communication between ports 437a, 437b and an interior of cover 420 (for example, an interior defined by top portion 423a and wall 423b). Cover 420 can be made of any of the materials described above with respect to cover 120, for example. Cover 420 can be transparent or semi-transparent and/or can be made of a more rigid material than membrane 462 of hub 460.

As also shown in FIG. 4J, cover 420 can include an opening 433 that allows a fluid tube to pass through cover 420 and to catheter hub 442 (for example, via connector 443). Cover 420 can include a connector 431 that can be similar to connector 131 in some respects. Opening 433 extends through wall 423b and connector 431, and can be sized and/or shaped to correspond to a size and/or shape of fluid tube connector 445 (which may also be referred to as a “fitting”) to inhibit (for example, prevent) rotation of connector 445, as further described below. As also further described below, inhibiting rotation of connector 445 within opening 433 inhibits rotation of tubes 449, 447 that may be bonded to connector 445 (and/or any or all other components of extension set 440) as described further below. Inhibiting rotation of tubes 449, 447 can advantageously inhibit rotation of connected components when catheter stabilization device 400 is in use, such as connector 443, catheter hub 442, and catheter 441.

Cover 420 can include one or more features to allow for securement to hub 460. As shown in at least FIGS. 4H-4J, cover 420 can include a protrusion 439a (see FIGS. 4H-4J) configured to secure to a latch arm 473a of hub 460 (see FIGS. 4Y-4Z and 4CC-4DD). In some implementations, cover 420 includes a recessed portion 439b (for example, at an end of cover 420) and protrusion 439a is positioned within such recessed portion 439b. Recessed portion 439b can be formed in wall 423b, for example. As shown in FIGS. 4J-4K, cover 420 can include a lip 497 having an opening 497a. Lip 497 can extend from a portion of cover 420, such as from an edge of wall 423b at an end of cover 420. Opening 497a can be configured to receive a protrusion 473b of hub 460 (see FIGS. 4Z and 4CC-4DD). Protrusion 439a of cover 420 can be configured to removably secure to latch arm 473a of hub 460. Protrusion 473b of hub 460 can be configured to removably secure within opening 497a. Securement between protrusion 439a and latch arm 473a and/or between protrusion 473b and opening 497a can allow cover 420 and hub 460 to be removably secured to one another. In some implementations, cover 420 can be disconnected from hub 460 by moving latch arm 473a out of engagement with protrusion 439a (and/or out of recessed portion 439b). After latch arm 473a is disengaged from protrusion 439a, cover 420 can be tilted relative to hub 460 such that protrusion 473b disengages from opening 497a. Cover 420 can then be removed from hub 460. In some implementations, cover 420 and hub 460 can form an air-tight seal around a catheter insertion site when device 400 is in use.

Cover 420 can be coupled (for example, movably coupled) to extension set 440 in a similar manner as that described in PCT. Pub. No. WO2022/140633A1 with reference to any of the cover and extension sets disclosed therein. FIG. 4G illustrates a perspective view of extension set 440 (along with cover 420, hub 460, catheter 441, and catheter hub 442) and FIG. 4R illustrates an enlarged view of a portion of extension set 440. Extension set 440 (which also may be referred to as a “fluid tube assembly”) can be utilized with and/or can interact with cover 420 and/or hub 460 in a manner that is similar to that described with respect to the extension sets in PCT. Pub. No. WO2022/140633A1. Extension set 440 can include a connector 443, a connector 445 (which may also be referred to as a “fitting”), a first fluid tube 449, a connector 446, a second fluid tube 447, a connector 448, and a sealing member 445g. Connectors 443, 448 and tube 447 can be similar or identical to connectors 143, 148 and tube 147 (respectively) described above. Connector 443 (which can be a male luer connector) can be configured to secure to catheter hub 442 to facilitate fluid communication between catheter hub 442 (and connected catheter 441) and tubes 449, 447. Connector 448 (which can be a female luer connector) can be configured to connect to a fluid delivery device. Connector 446 can be configured to removably secure to fitting 445, for example, via engagement between threads within connector 446 and threads 445b on fitting 445. Connector 446 can be secured to fitting 445: after fitting 445 is positioned within opening 433; after connector 443 is secured to catheter hub 442; and/or after cover 420 is secured to hub 460 (see FIG. 4F). FIG. 4S shows connector 446 not secured to fitting 445 while FIG. 4R illustrates connector 446 secured to fitting 445 (without also showing cover 420). FIG. 4F illustrates connector 446 and fitting 445 secured to one another while also secured to cover 420. FIG. 4T illustrates a side view of fitting 445 and FIGS. 4U-4V illustrate perspective views of fitting 445.

Fitting 445 can fluidly couple tubes 447 and 449 together. Fitting 445 can include a first interior portion 445c configured to receive tube 447 and a second interior portion 445e configured to receive tube 449. Tubes 447, 449 can be permanently secured (for example, bonded) to fitting 445, for example, within such interior portions 445c, 445e. Fitting 445 can include an inner wall 445d that separates interior portions 445c, 445e (see FIGS. 4U-4V and 4F). Inner wall 445d can act as an abutment surface for ends of tubes 447, 449 (see FIG. 4F). Inner wall 445d can include an opening (for example, the circular opening shown in FIG. 4V) that can facilitate fluid communication between tubes 447, 449. Fitting 445 can include one or more (for example, a plurality) of features that are configured to mate with corresponding features within opening 433 of cover 420. Such corresponding mating features can inhibit (for example, prevent) rotation of fitting 445 relative to opening 433 and cover 420. For example, fitting 445 can include one or a plurality of ribs 445a (see FIGS. 4R-4U) that are sized and shaped to match a size and shape of one or a plurality of grooves extending within opening 433/connector 431 (see 4J-4K). Such corresponding mating features can inhibit rotation of fitting 445 relative to cover 420, which in turn inhibits rotation of components of extension set 440 relative to cover 420. This advantageously can in turn inhibit rotation of catheter hub 442 and/or catheter 441 when extension set 440 is connected to catheter hub 442 (for example, via connector 443), which inhibits movement of catheter 441 within the subject's vasculature and at the catheter insertion site. Extension set 440 can include a sealing member 445g, for example, an O-ring that can be held in place by a rim 445f at an end of fitting 445. Sealing member 445g can seal opening 433 of cover 420 (for example, at an interior portion of cover 420) when extension set 440 and cover 420 are secured to one another, for example, as shown in FIG. 4F.

As discussed above, extension set 440 can include two fluid tubes, tube 447 and tube 449. Inclusion of both of fluid tubes 449 and 447 in extension set 440 provides multiple advantages. FIG. 4F illustrates a cross-section taken through cover 420, hub 460, and extension set 440 when secured together and to catheter hub 442. In some implementations, fluid tube 449 is made of a material that is flexible. This advantageously allows tube 449 to be able to bend and accommodate movement of catheter hub holder 474 (and connected catheter hub 442) as described above, even when extension set 440 is secured to cover 420 and hub 460. In some implementations, fluid tube 449 is made of a material that is more flexible than a material that fluid tube 447 is made of. In some implementations, tube 447 is more durable than tube 449. This can advantageously allow tube 447 to withstand forces and/or stresses (for example, due to pulling or twisting forces) that tube 447 may be exposed to in the environment surrounding the catheter stabilization device 400 and catheter insertion site on the subject.

Catheter stabilization device 400 can be utilized at a catheter insertion site to secure catheter hub 442 when catheter 441 is inserted in the catheter insertion site. Catheter stabilization device 400 can secure to skin on a portion of a subject's body (such as an arm) and also secure to such catheter hub 442. The securement to catheter hub 442 allows device 400 to stabilize (for example, operably position) catheter 441 with respect to the catheter insertion site and/or a subject's vein in which catheter 441 extends. In some cases, movement of catheter stabilization device 400 (and/or skin to which device 400 is attached) may cause catheter 441 to move relative to the catheter insertion site and/or the vein in which catheter 441 extends. As discussed previously, it may be desirable to inhibit relative movement between catheter 441 and the subject's vein and/or the insertion site. Catheter stabilization device 400 can include a first portion that is configured to secure to the subject's skin and a second portion that is configured to secure to catheter hub 442 connected to catheter 441. Such first and second portions can be movably coupled to one another to allow for relative movement between the first portion and catheter hub 442 and catheter 441. Membrane 462, frame 464, and/or clamp 466, described below, can be implementations of such first portion. Membrane 462′, frame 464′, and/or clamp 466′, described further below, can also be an implementation of such first portion. Membrane 462″, frame 464′', and/or clamp 466″, described further below, can also be an implementation of such first portion. Catheter hub holders 474, 474′, 474″, also described below, can each be an implementation of such second portion.

FIGS. 4W-4X illustrate top perspective views of hub 460 secured to catheter hub 442 and catheter 441. FIGS. 4Y-4RR illustrate hub 460 or portions thereof. Hub 460 (which may also be referred to as a “dock” or “base”) can be similar to hub 160 in some or many respects. Hub 460 can include a frame 464 and a membrane 462. In some implementations, frame 464 comprises a material that is more rigid than membrane 462. In some implementations, membrane 462 is overmolded onto frame 464, for example, in a similar manner as that described in PCT. Pub. No. WO2022/140633A1 with respect to any of the membranes and frames disclosed therein. In some implementations, membrane 462 is an adhesive layer (for example, coupled with a foam layer) that is configured to secure to skin and configured to be secured to frame 464. Frame 464 can include a frame body 470 and a latch arm 473a extending from frame body 470. Frame body 470 can be similar to frame body 170. Latch arm 473a can be configured to secure (for example, removably secure) to protrusion 439a of cover 420 as discussed above. In some implementations, latch arm 473a (for example, an end of latch arm 473a) is sized and/or shaped to fit within the recessed portion 439b of cover 420 so as to minimize or eliminate the possibility that latch arm 473a inadvertently catches wires, tubes, clothing, or other objects in the vicinity of around catheter stabilization device 400 when in use on a subject and/or in a medical environment. In some implementations, membrane 462 is an adhesive layer (for example, coupled with a foam layer) that is configured to secure to skin and configured to be secured to frame 464. Membrane 462 can include one or more or a plurality of perforations 462a that can be identical to perforations 162a. Membrane 462 can be similar or identical to membrane 162 in some or all respects.

Frame 464 can include one or more indicators extending from frame body 470 that can aid in positioning hub 460 relative to a catheter insertion site and/or relative to catheter 441 and/or catheter hub 442. For example, frame 464 can include fingers 475 extending outward from a surface of frame body 470. As shown, fingers 475 can extend from opposing sides of frame body 470 toward one another. Fingers 475 can have a first end connected to a portion of frame body 470 and a second end that is “free”. The free ends of fingers 475 can be spaced from one another by a gap. Such gap can allow the catheter insertion site to be viewed and positioned therewithin, for example, when hub 460 is secured to the subject's skin. In some implementations, such free ends of fingers 475 are tapered. In some implementations, hub 460 (for example, frame 464, membrane 462, and/or cover 420 are made of a transparent material. In some implementations where fingers 475 are made of a transparent material, free ends of fingers 475 are painted with a color that more easily allows visibility of such free ends and therefore aids alignment and/or positioning of the hub 460 (and fingers 475) relative to a catheter insertion site. As mentioned elsewhere herein, membrane 462 can be overmolded to frame 464. In some implementations, frame 464 includes one or more features that can facilitate bonding of membrane 462 to frame 464. For example, with reference to FIG. 4HH, frame 464 can include one or a plurality of grooves 499 in frame body 470 that can facilitate bonding of membrane 462. Such grooves 499 can be spaced from one another and positioned on an outer surface of frame body 470. In some implementations, grooves 499 are arranged along an entirety of a perimeter of frame body 470.

FIGS. 4W-4X illustrate top perspective views of hub 460 with catheter hub 442 secured thereto and FIGS. 4Y-4Z illustrate top perspective views of hub 460 without catheter hub 442. FIG. 4AA illustrates a top view, FIG. 4BB illustrates a bottom view, FIGS. 4CC-4DD illustrate side views, FIG. 4EE illustrates a front view, and FIG. 4FF illustrates a rear view of hub 460. As mentioned previously, hub 460 can include a frame 470 and a membrane 462. Hub 460 also can include a clamp 466 (which also may be referred to as a “brace” or a “boom”) and a catheter hub holder 474 (which may also be referred to as a “catheter hub retainer”). Frame 464, membrane 462, and/or clamp 466 (which can be connected to one another) can be configured to secure (for example, removably secure) to the subject's skin (for example, via membrane 462. Catheter hub holder 474 can be configured to secure to catheter hub 442. As described in more detail below, catheter hub holder 474 can be movably coupled (for example, rotatably coupled) to membrane 462 and/or frame 464 via clamp 466.

Clamp 466 can be connected to one or more portions of frame 464. For example, where frame 464 has an annular-shaped frame body 470, clamp 466 can be connected to opposite sides of frame body 470, as shown in the figures. In implementations of catheter stabilization device 400 including both of frame 464 and membrane 462 and in which membrane 462 includes opening 463, clamp 466 can extend across and/or over opening 463 between two portions of frame body 470. In some implementations, clamp 466 is configured to removably connect to frame 464 (for example, frame body 470). For example, clamp 466 can include a first end configured to connect to a first portion of frame body 470 and a second end configured to connect to a second portion of frame body 470. In some implementations, clamp 466 is pivotably connected to frame 464 (for example, frame body 470) and is configured to pivot between a plurality of positions, as further described below.

FIGS. 4HH-4KK show, among other things, frame 464 and frame body 470 without membrane 462. FIGS. 4OO-4RR illustrate top and bottom perspective views of clamp 466. In some implementations, clamp 466 includes a pin 466a (for example, at a first end of clamp 466). Pin 466a can be configured to be connected to a portion of frame body 470 in a manner that allows clamp 466 to be pivoted relative to frame 464. Frame 464 can be configured to pivotably secure pin 466a. For example, with reference to FIG. 4JJ (which illustrates an enlarged portion of frame 464), frame 464 can include a hook 465a and/or one or both of protrusions 465b that can be configured to receive pin 466a such that pin 466a is secured but is allowed to pivot about an axis extending through pin 466a (which may be referred to as a “pin axis”). FIGS. 4W-4HH illustrate clamp 466 in a first position and FIG. 4II illustrates clamp 466 in a second position. Clamp 466 can be configured to be pivoted between a plurality of rotational positions (about an axis extending through pin 466a), including but not limited to the positions shown in the figures. FIG. 4II may be an example of an “open” position of the clamp 466 that can be utilized prior to securement of catheter hub 442 to catheter hub holder 474. In some implementations, clamp 466 includes one or more bumps 466g extending outward from pin 466a (see FIGS. 4PP-RR). Bumps 466g can contact and/or engage protrusions 465b of frame 464 when clamp 466 is rotated (for example, between the positions shown in FIGS. 4HH-4II). Bumps 466g can at least partially inhibit rotation of clamp 466 and/or can provide tactile feedback when clamp 466 is pivoted.

Clamp 466 can include one or more mechanical engagement features configured to allow clamp 466 to secure to frame body 470. For example, with reference to FIGS. 4OO-4RR, clamp 466 can include a latch portion 466b (which also may be referred to as a “latch”) that can secure to latch protrusion 467a extending from frame body 470. Latch portion 466b can be arranged at an end of clamp 466, for example, an end opposite to the end on which pin 466a is arranged. In some implementations, when latch 466b is secured to frame body 470, a portion of latch 466b passes over protrusion 467a such that protrusion 467a rests against and/or contacts a ledge 469a of latch 466b. With reference to FIG. 4GG, protrusion 467a can inhibit disconnection of latch portion 466b from frame 464, for example, inhibit clamp 466 from pivoting towards the position shown in FIG. 4II. In some implementations, latch portion 466b can include one or both of nubs 469b, 469c and frame 464 can include one or both of protrusions 467c, 467d which can extend from frame body 470. Protrusions 467c, 467d can limit and/or inhibit movement of latch portion 466b in a direction that is parallel to a side of frame 464, for example in a direction that is parallel to an axis extending along a width w₂ of hub 460 (see FIG. 4AA). Protrusions 467c, 467d (which may be referred to as “guide protrusions” or “alignment protrusions”) can help guide latch portion 466b into engagement with protrusions 467a, 467b. With reference to FIG. 4KK and FIGS. 4OO-4QQ, nubs 469b, 469c can be positioned adjacent to and/or can contact protrusions 467b that extend from frame body 470 when latch portion 466b is secured to frame 464 in the position shown in FIG. 4HH (which may be referred to as a “closed” position). In some implementations, latch portion 466b can be detached from frame 464 body by compressing clamp 466 (for example, inward).

Pivotable connection of clamp 466 to frame 464 (for example, via pin 466a) advantageously allows for a catheter hub 442 to be efficiently and conveniently secured to catheter hub holder 474. For example, clamp 466 can be moved from a position such as that shown in FIG. 4II (which is an example of an “open” position of clamp 466) to a position such as that shown in FIG. 4HH (which is an example of a “closed position” of clamp 466) after catheter 441 is inserted into an insertion site and hub 460 is secured to the subject's skin around the insertion site. In such scenario, clamp 466 can be pivoted to a closed position in a manner that causes catheter hub holder 474 to secure to catheter hub 442. Pivoting clamp 466 to a closed position (for example, a position where latch portion 466a is secured to frame body 470) can move catheter hub holder 474 closer to the subject's skin and catheter hub 442. Catheter hub holder 474 and the securement of catheter hub holder 474 to catheter hub 442 is described further below.

Although the figures and description above illustrate and describe implementations of clamp 466 that includes pin 466a and latch portion 466b, other implementations of clamp 466 are possible. For example, in some variants, clamp 466 does not include pin 466 but may still be configured to removable connect to frame 464, for example, in a manner that allows clamp 466 to cause catheter hub holder 474 to secure to catheter hub 442. For example, in some variants clamp 466 is configured to secure to two portions of frame body 470 (for example, at or near the locations shown in the figures) via a connection (such as a press-fit and/or snap-fit connection). For example, clamp 466 can be configured to have latch portion 466b at both ends thereof, instead of having pin 466a at one end and latch portion 466b at the other end of clamp 466. In such variants, frame 464 can include protrusions 467a, 467b, 467c, and/or 467d on both sides of frame body 470 so as to secure to both latch portions 466b on each end of clamp 466. In such variants, clamp 466 (and catheter hub holder 474) can be secured to frame body 470 over opening 463 and overtop catheter hub 442 such that catheter hub holder 474 secures to catheter hub 442. In some variants, clamp 466 can be removably connectable to such portions of frame body 470.

As discussed above, hub 460 can include a catheter hub holder 474. As also discussed above, catheter hub holder 474 can be configured to secure to catheter hub 442. FIGS. 4LL-4MM illustrate perspective views and FIG. 4NN illustrates a front view of catheter hub holder 474 alone, while FIGS. 4Y-4II illustrate catheter hub holder 474 connected to clamp 466. Catheter hub holder 474 can be configured to secure to catheter hub 442. For example, catheter hub holder 474 can include a body 474a configured to secure to catheter hub 442. In some implementations, body 474a comprises a C-shape. In some implementations, body 474a is configured to surround at least about 25%, at least about 50%, or at least about 75% of a cross-section of catheter hub 442. In some implementations, body 474a is shaped to secure around a portion of a cylindrical section of catheter hub 442. In some implementations, body 474a is shaped to surround less than an entire perimeter of a cross-section of catheter hub 442 (for example, a circular cross-section of catheter hub 442). In some implementations, body 474a is configured so as to not allow relative movement between body 474a and catheter hub 442 when secured together. In some implementations, body 474a is configured to inhibit inadvertent disconnection between catheter hub holder 474 and catheter hub 442. Catheter hub holder 474 can additionally be coupled to clamp 466. For example, catheter hub holder 474 can be movably coupled (for example, rotatably coupled) to clamp 466. Catheter hub holder 474 can be movably coupled (for example, rotatably coupled) to frame 464 and/or membrane 462 via clamp 466. Catheter hub holder 474 can be coupled to frame 464 and/or membrane 462 (for example, via clamp 466) via a gimbal arrangement. Catheter hub holder 474 can be rotatably coupled to frame 464 and/or membrane 462 (for example, via clamp 466) such that catheter hub holder 474 is able to rotate at least partially about one or more of three perpendicular axes (for example, at least one, at least two, or all three of such axes). In some implementations, catheter hub holder 474 comprises a ball 474c. Ball 474c can be secured within a cavity of clamp 466. For example, clamp 466 can include a cavity 466c (see FIGS. 4OO-4RR) that is sized and/or shaped to accommodate ball 474c. FIGS. 4SS-4TT illustrate three axes 401, 403, and 405 which are perpendicular to one another. With reference to FIGS. 4SS-4TT and FIG. 4AA, a width w₂ of hub 460 extends along axis 405 and a length L₂ of hub 460 extends along axis 403. A height of hub 460 (and/or catheter stabilization device 400) extends along axis 401. Cavity 466c can be configured to allow ball 474c to rotate at least partially about one or more of axes 401, 403, and/or 405. Ball 474c can comprise a spherical shape. In some implementations, ball 474c and/or surface(s) defining cavity 466c comprise smooth surfaces to facilitate rotation of ball 474c within cavity 466c. In some implementations, catheter hub holder 474 includes a stem 474b positioned in between body 474a and ball 474c (see FIGS. 4LL-4N). In some implementations, body 474a includes a planar portion (which can be connected to two curved legs of body 474a) to which stem 474b connects.

With reference to FIGS. 4LL-4NN, in some implementations, catheter hub holder 474 includes one or more protrusions 474d (which may be referred to as “ears”) extending from ball 474 (for example, radially outward). In some implementations, catheter hub holder 474 includes both of protrusions 474d. With reference to FIGS. 4QQ-4RR, clamp 466 can include one or more slots 466d configured to receive protrusion(s) 474d. FIG. 4SS illustrates a cross-section taken through a portion of hub 460, as indicated in FIG. 4AA. Protrusions 474d can advantageously limit an amount of rotation of catheter hub holder 474 relative to clamp 466 (and frame 464 and/or membrane 462). Accordingly, protrusions 474d can limit a degree of rotation of catheter hub 442 when secured to catheter hub holder 474, which can advantageously facilitate proper positioning and/or alignment of the catheter hub 442 and catheter 441 when inserted at the catheter insertion site. With reference to at least FIGS. 4SS and 4TT, protrusions 474d can limit an amount of rotation of ball 474c, catheter hub holder 474, and a connected catheter hub 442 about each of axes 401, 403, and 405. In some implementations, protrusions 474d can limit rotation of ball 474c, catheter hub holder 474, and/or a connected catheter hub 442 relative to any or all of axes 401, 403, and 405 to an amount that is between about 0 degrees and about 60 degrees, between about 0 degrees and about 55 degrees, between about 0 degrees and about 50 degrees, between about 0 degrees and about 45 degrees, between about 0 degrees and about 40 degrees, between about 0 degrees and about 35 degrees, between about 0 degrees and about 30 degrees, between about 0 degrees and about 25 degrees, between about 0 degrees and about 20 degrees, between about 0 degrees and about 15 degrees, between about 0 degrees and about 10 degrees, or between about 0 degrees and about 5 degrees. In some variants, catheter hub holder 474 does not include protrusions 474d.

In some implementations, clamp 466 includes one or more features to limit an amount of rotation of ball 474c, catheter hub holder 474, and/or a connected catheter hub 442 about any or all of axes 401, 403, and 405. For example, with reference to FIGS. 4QQ-4RR, clamp 466 can include one or more protrusions 466e, 466f extending from a surface of clamp 466, for example, a surface proximate cavity 466c. With reference to FIG. 4SS, protrusions 466e, 466f can limit rotation of catheter hub holder 474 relative to axis 403 by contacting body 474a of catheter hub holder 474 when catheter hub holder 474 is rotated a certain amount. For example, protrusions 466e, 466f can be configured (for example, sized and/or shaped) such that they limit rotation of catheter hub holder 474 about axis 403 (by contacting body 474a) to any of the values or ranges mentioned above with respect to protrusions 474d. In some implementations, protrusions 466e have a greater length than protrusions 466f. This can advantageously maintain an inclination angle of body 474a relative to the subject's skin, for example, within a certain range. Protrusions 466e, 466f can have a cylindrical cross-section or a different cross-section. In some variants, clamp 466 does not include protrusions 466e and/or does not include protrusions 466f.

With reference to FIGS. 4LL-4NN, in some implementations, catheter hub holder 474 includes one or both of arms 474e extending outward from ends of body 474a. Such arms 474e can have a curved shape. Arms 474e can extend from ends of curved legs of body 474a. Arms 474e can extend in an opposite direction from one another. Arms 474e can advantageously facilitate positioning and/or alignment of catheter hub 442 relative to body 474a during securement. For example, when clamp 466 is pivoted from an open position (such as that shown in FIG. 4II) to a closed position (such as that shown in FIG. 4HH) overtop catheter hub 442, one or both of arms 474e can contact, push, and/or align catheter hub 442 with body 474a such that catheter hub 442 is engaged by and positioned within body 474a.

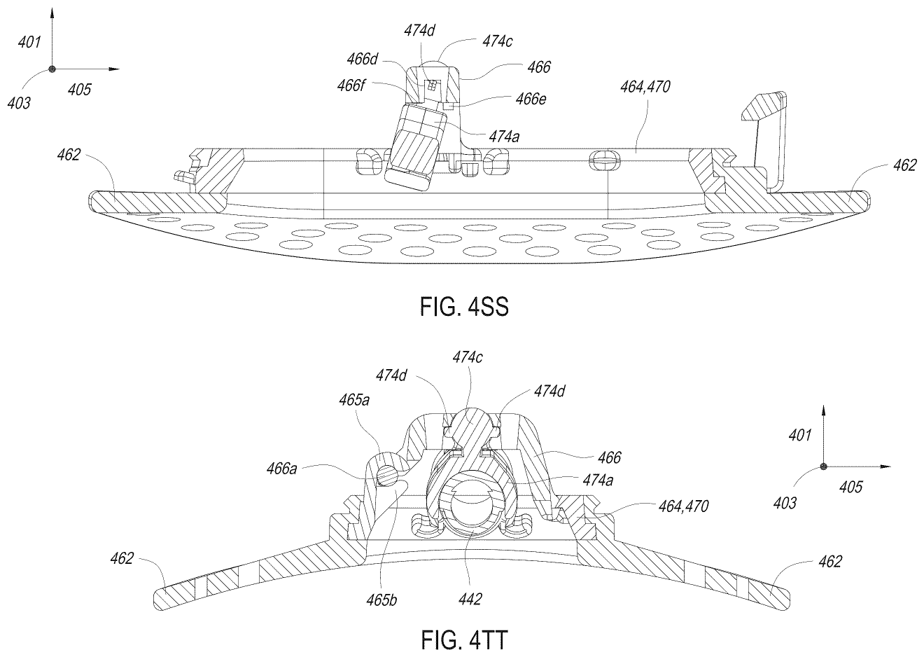

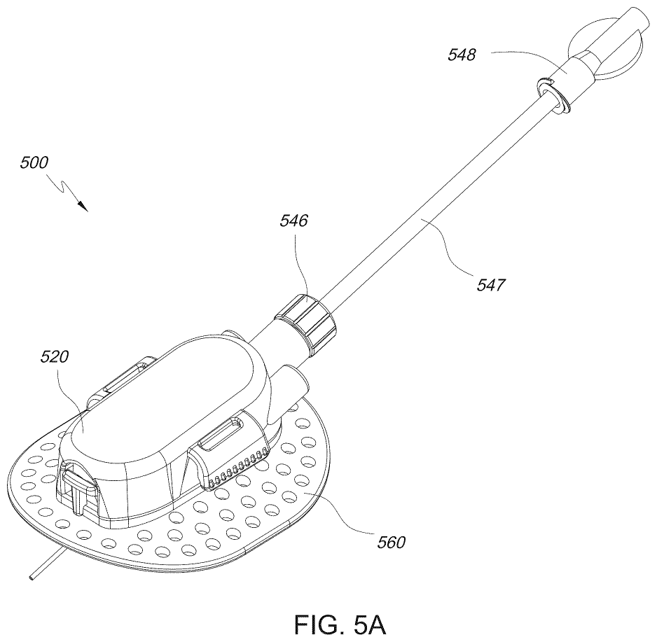

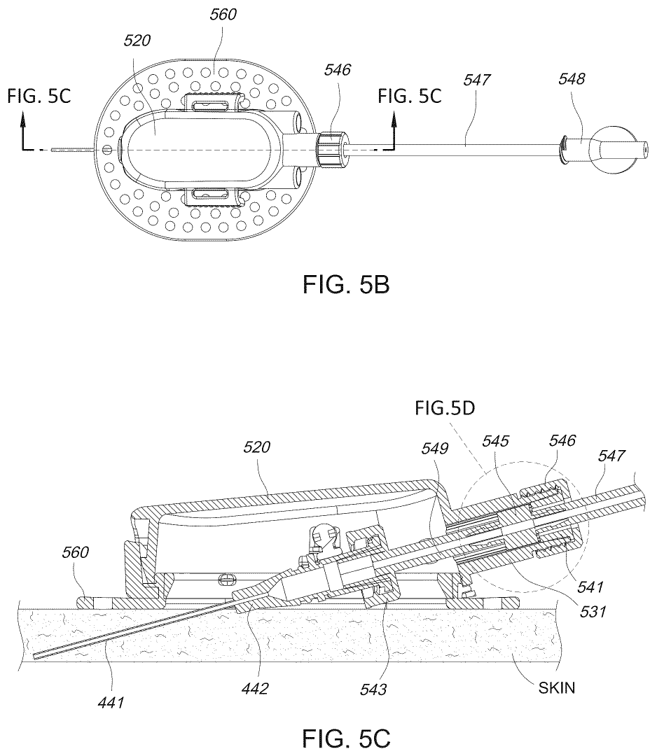

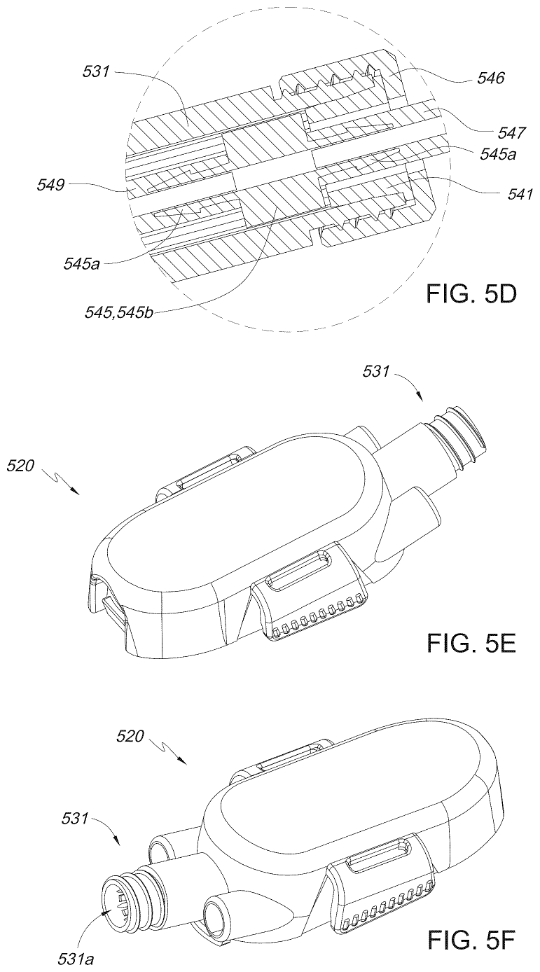

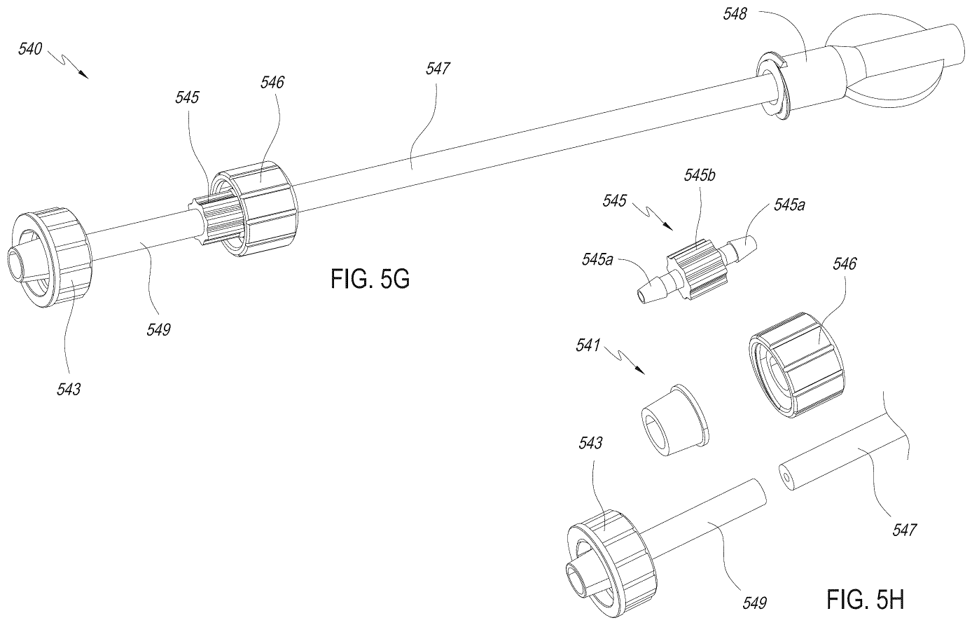

FIGS. 5A-5H illustrate another implementation of a catheter stabilization device 500 or portions thereof. Catheter stabilization device 500 can be similar or identical to catheter stabilization device 400 in some or many respects. FIGS. 5A and 5B illustrate top perspective and top views (respectively) of catheter stabilization device 500. FIG. 5C illustrates a cross-sectional view taken through device 500 in an example condition of use. Although not illustrated in FIG. 5C, it is to be understood that catheter 441 can be at least partially positioned within a vein of the subject when in use. Catheter stabilization device 500 can include a cover 520, a hub 560, and an extension set 540. Hub 560 can be similar or identical to hub 460. FIGS. 5D-5E illustrate top perspective views of cover 520. Cover 220 can be identical to cover 420 except with respect to connector 531. Connector 531 includes a threaded end that is exposed and that is configured to secure to threads of connector 546 (see FIG. 5C). FIG. 5G illustrates extension set 540, which can include connector 543, tube 549, tube 547, connector 548, connector 546, fitting 545, and gasket 541. Connector 543 can be similar or identical to connector 443, connector 548 can be similar or identical to connector 448, tubes 547, 549 can be similar or identical to tubes 447, 449, and connector 546 can be similar or identical to connector 446. Although FIGS. 5G-5H illustrate connector 543 attached to tube 549, it is to be understood that tube 549 can be a separate component from connector 543 and can be later bonded to a portion of connector 543.

Connector 545 can fluidly couple tubes 547, 549 together. Connector 545 can include ends 545a that may be tapered (for example, barbs). Ends 545a can be secured within ends of tubes 547, 549. Connector 545 can include one or more (for example, a plurality) of features that are configured to mate with corresponding features within an opening extending through cover 520 (for example, connector 531) and which inhibit (for example, prevent) rotation of connector 545 relative to cover 520. For example, connector 545 can include one or a plurality of ribs 545b that are sized and shaped to match a size and shape of one or a plurality of grooves 531a extending within connector 531. Such rib(s) 545b can be similar or identical to ribs 445a discussed above.

FIG. 5D illustrates an enlarged view of a portion of that shown in FIG. 5C. FIG. 5D illustrates extension set 540 (which also may be referred to as a “fluid tube assembly”) connected to cover 520, via connection between connector 546 and threaded end of connector 531. In some implementations, extension set 540 includes a gasket 541 that is configured to engage tube 547 and inhibit (for example, prevent) longitudinal movement of tube 547 (and thus, extension set 540) when connector 546 secures to connector 531, for example, in a similar manner as that described above with respect to gasket 149 of extension set 140.

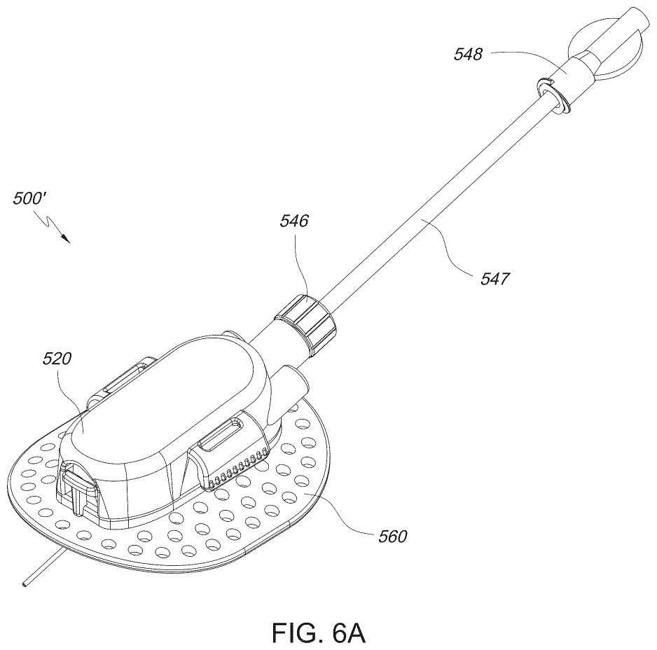

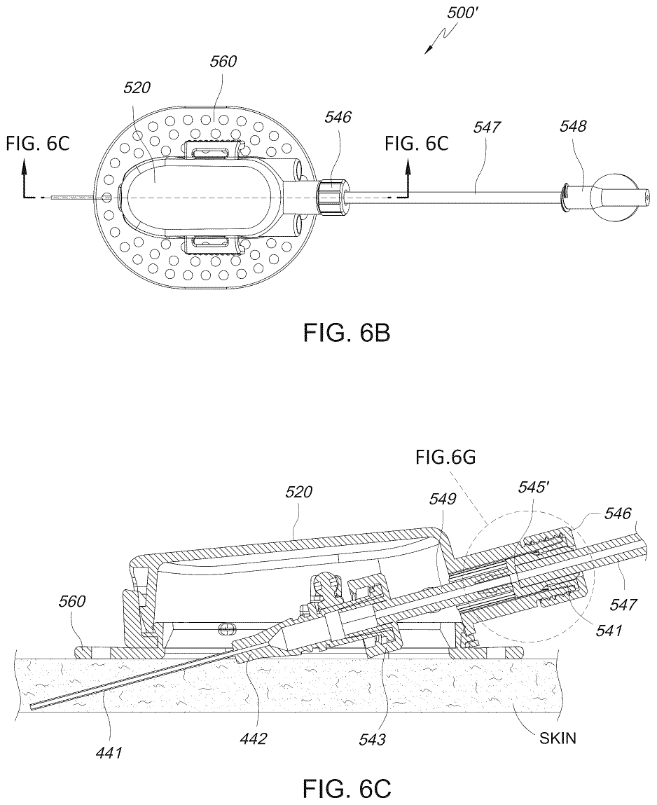

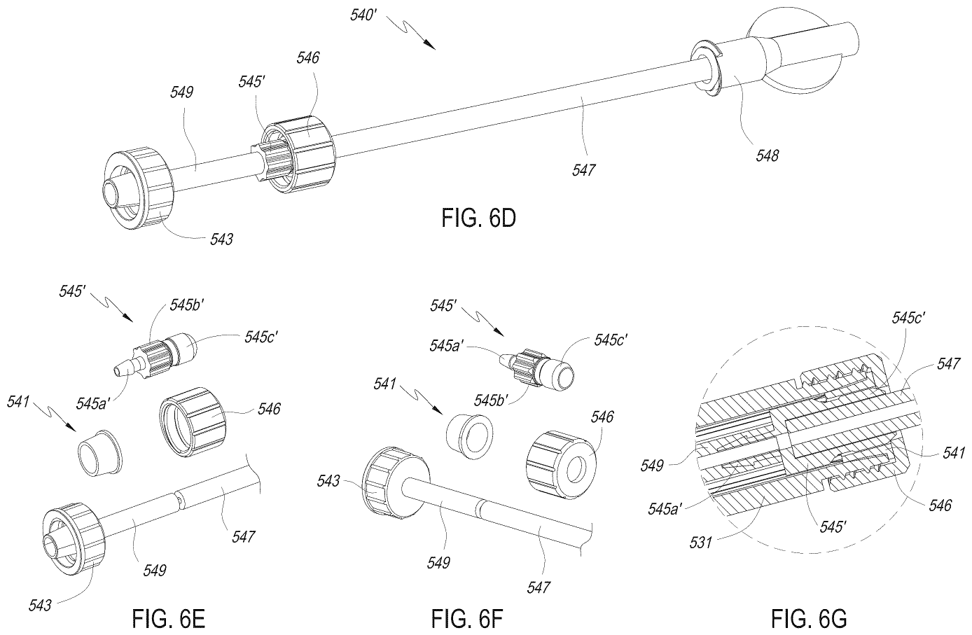

FIGS. 6A-6G illustrate another implementation of a catheter stabilization device 500′ or portions thereof. Catheter stabilization device 500′ is identical to catheter stabilization device 500 except with respect to extension set 200′. Catheter stabilization device 500′ can include cover 520 and hub 560. FIGS. 6A-6B illustrate top perspective and top views of device 500′. FIG. 6C illustrates a cross-sectional view taken through device 500′ in an example condition of use. Although not illustrated in FIG. 6C, it is to be understood that catheter 441 can be at least partially positioned within a vein of the subject when in use. FIGS. 6D-6F illustrate extension set 540′, which can include connector 543, tube 549, tube 547, connector 548, connector 546, fitting 545′, and gasket 541. Fitting 545′ can fluidly couple tubes 547, 549 together. Fitting 545′ can include a first end 545a′ that can be tapered configured to be secured within an end of tube 549 and a second end 545c′ having a cavity configured to receive an end of tube 547. Fitting 545′ can be permanently secured to tubes 549, 547. Fitting 545′ can include one or more (for example, a plurality) of features that are configured to mate with corresponding features within an opening extending through cover 520, for example, connector 531 and which inhibit (for example, prevent) rotation of connector 545′ relative to cover 520. For example, connector 545′ can include one or a plurality of ribs 545b′ similar or identical to ribs 445a, and/or 545a.

FIG. 6G illustrates an enlarged view of a portion of that shown in FIG. 6C. FIG. 6G illustrates extension set 540′ connected to cover 520, via connection between connector 546 and threaded end of connector 531. In some implementations, extension set 540′ includes a gasket 541 that is configured to engage tube 547 and inhibit (for example, prevent) longitudinal movement of tube 547 (and thus, extension set 540′) when connector 546 secures to connector 531, for example, in a similar manner as that described above with respect to gasket 149 of extension set 140.

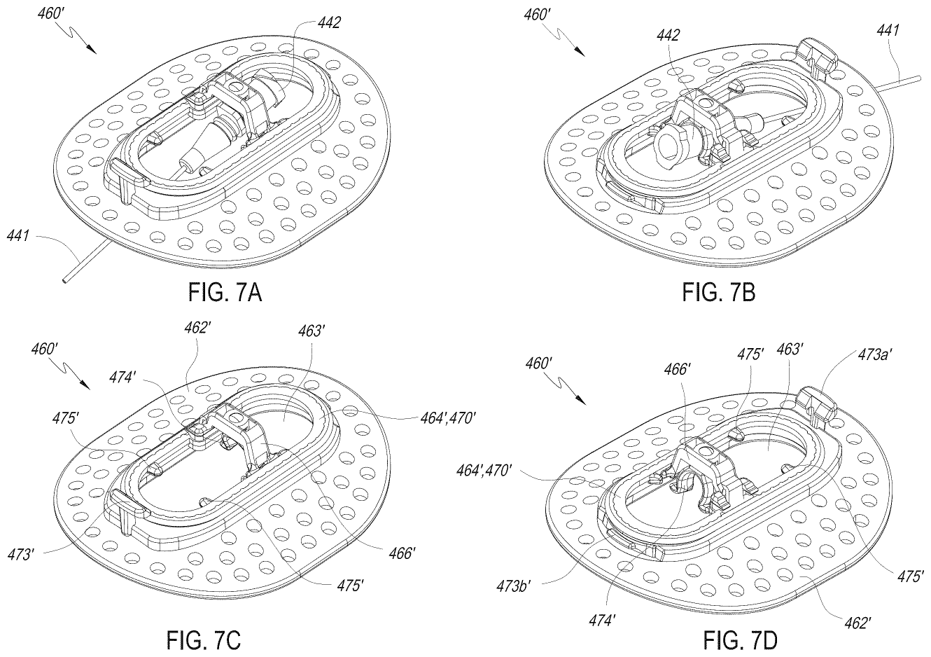

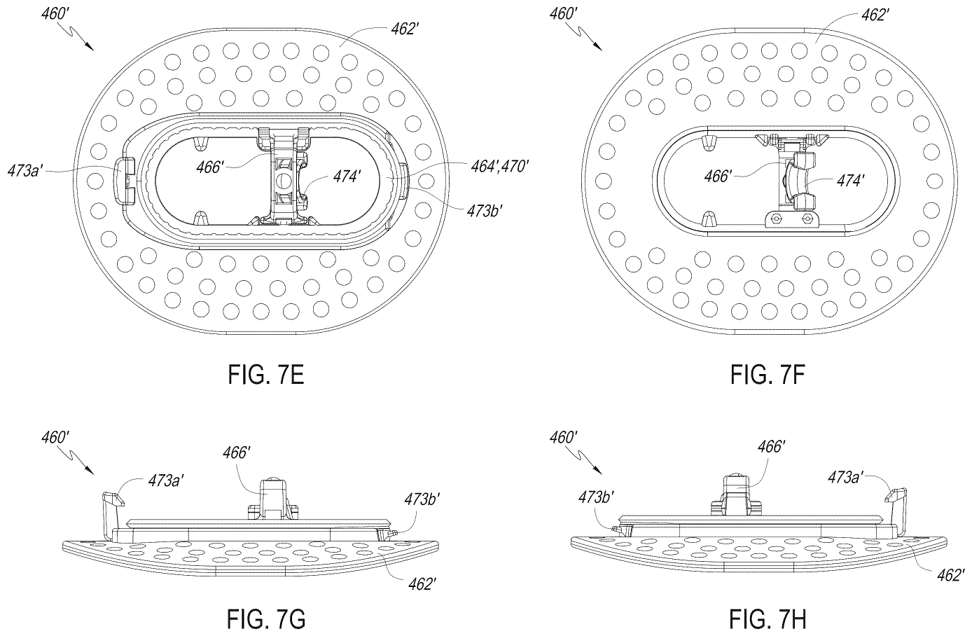

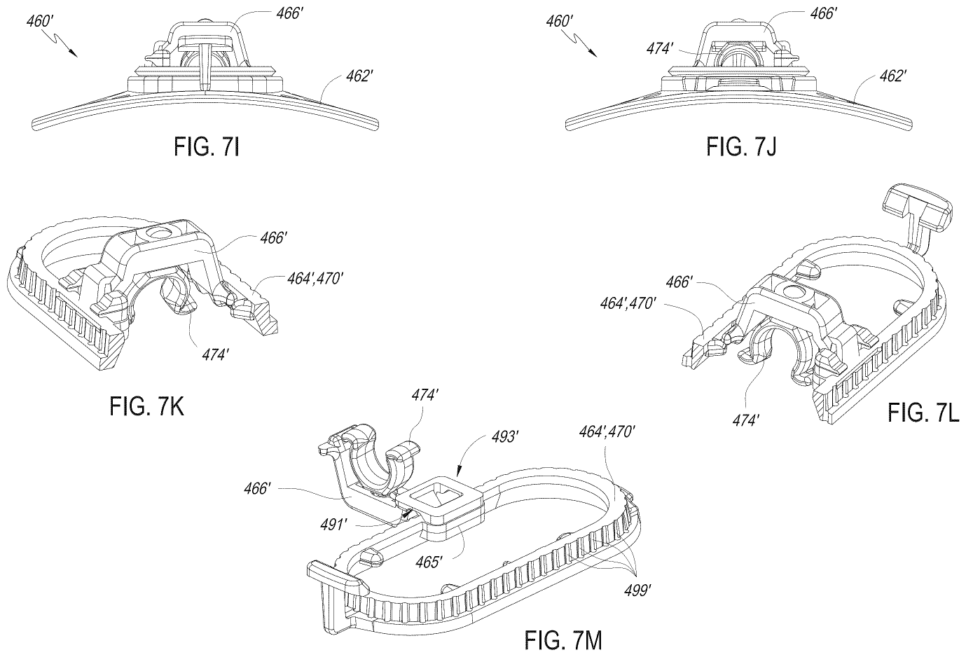

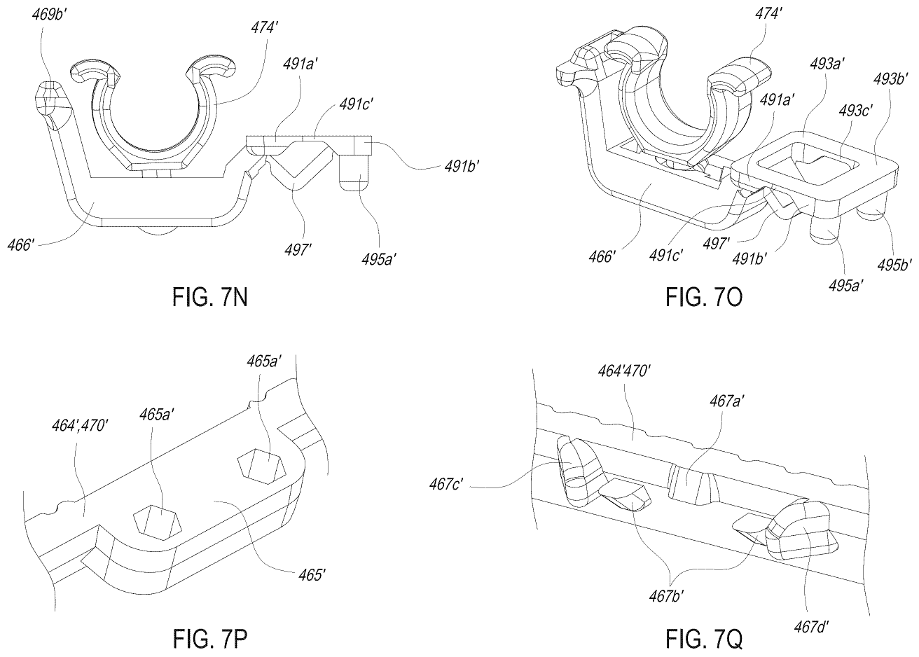

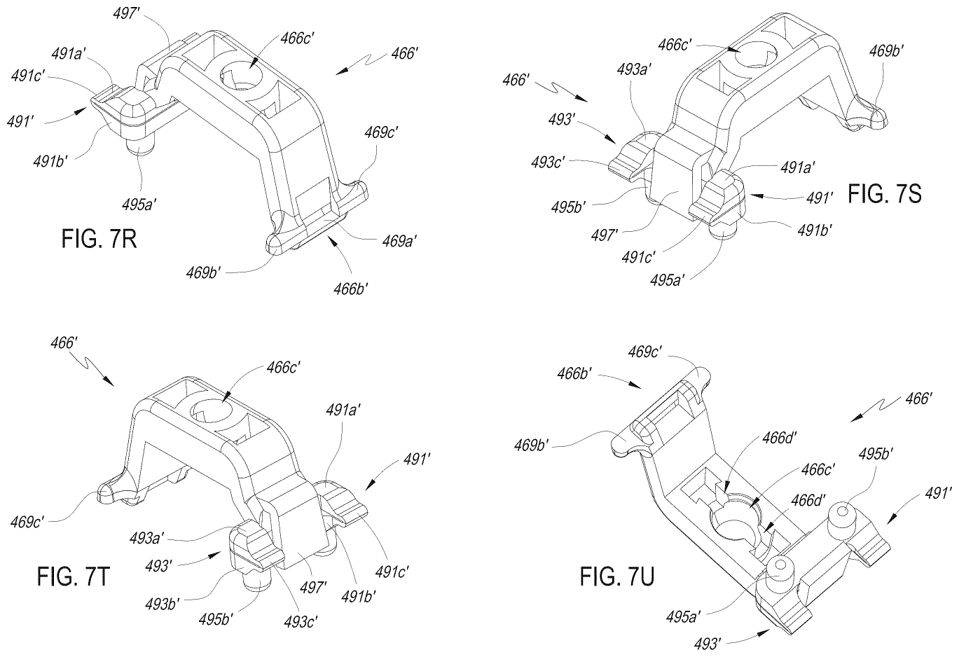

FIGS. 7A-7U illustrate another implementation of a hub 460′ that can be utilized with cover 420 and extension set 440 in a similar manner as that shown and/or described herein with respect to hub 460. Hub 460′ (which also may be referred to as a “dock” or “base”) can also be utilized with cover 520 and any of extension sets 540, 540′. FIGS. 7A-7B illustrate top perspective views of hub 460′ with catheter hub 442 secured thereto, while FIGS. 7C-7D illustrate the same views as FIGS. 7A-7B but without catheter hub 442 connected thereto. FIGS. 7E-7F illustrate top and bottom views of hub 460′ (respectively). FIGS. 7G-7H illustrate side views of hub 460′. FIGS. 7I-7J illustrate front and rear views (respectively) of hub 460′. Hub 460′ can include a membrane 462′ that can be similar or identical to membrane 462. For example, membrane 462′ can have an opening 463′ that can be similar or identical to opening 463 of membrane 462. Hub 460′ can also include a frame 464′, which can be similar to frame 464 in some or many respects. For example, frame 464′ can have an annular-shaped frame body 470′, and can have fingers 475′ that can be similar or identical to fingers 475. Hub 460′ can include latch arm 473a′ that can be similar or identical to latch arm 473. Hub 460′ can include a protrusion 473b′ that can be similar or identical to protrusion 473b of hub 460. Hub 460′ can include a clamp 466′ (which also may be referred to as a “brace” or a “boom”) and a catheter hub holder 474′. Catheter hub holder 574′ can be similar or identical to catheter hub holder 474. With reference to FIG. 7M, frame 464′ can include one or a plurality of grooves 499′ in frame body 470′ that can facilitate bonding of membrane 462′ and which can be similar or identical to grooves 499.

Frame 464′, membrane 462′, and/or clamp 466′ (which can be connected to one another) can be configured to secure (for example, removably secure) to the subject's skin (for example, via membrane 462′). Catheter hub holder 474′ can be configured to secure to catheter hub 442. As described in more detail below, catheter hub holder 474′ can be movably coupled (for example, rotatably coupled) to membrane 462′ and/or frame 464′ via clamp 466′ in a similar or identical manner as that described above with respect to catheter hub holder 474 and clamp 466. Clamp 466′ be connected to one or more portions of frame 464′. For example, where frame 464′ has an annular-shaped frame body 470′, clamp 466′ can be connected to opposite sides of frame body 470′, as shown in the figures. In implementations of catheter stabilization device 400 having frame 464′ and membrane 462′ and in which membrane 462′ includes opening 463′, clamp 466′ can extend across and/or over opening 463′ between two portions of frame body 470′.

FIGS. 7R-7T illustrate top perspective views of clamp 466′ and FIG. 7U illustrates a bottom perspective view of clamp 466′. Clamp 466′ can include a latch portion 466b′ that can be similar or identical to latch portion 466b of clamp 466. Latch portion 466b′ can be arranged at a first end of clamp 466′. Latch portion 466b′ can include one or more nubs 469b′, 469c′, and ledge 469a′, each of which can be similar or identical to nubs 469b, 469c, and ledge 469a of clamp 466. Latch portion 466b′ can be removably securable to a portion of frame 464′. With reference to FIG. 7Q (which illustrates an enlarged view of a portion of frame 464′), frame 464′ can include protrusions 467a′, 467b′, 467c′, 467d′ which can be similar or identical to protrusions 467a, 467b, 467c, 467d. Nubs 469b′, 469c′ and ledge 469a′ can interact with protrusions 467a′, 467b′, 467c′, 467d′ in a similar or identical manner as that described above with respect to nubs 469b, 469c and ledge 469a and protrusions 467a, 467b, 467c, 467d.

Clamp 466′ can be pivotably connected to frame 464′ and be configured to pivot between a plurality of positions in a similar manner as that described above with respect to clamp 466. FIGS. 7K-7M illustrate frame 464′, frame body 470′, clamp 466′, and catheter hub holder 474′ without membrane 462′. FIGS. 7N-70 illustrate side and bottom perspective views of clamp 466′ and catheter hub holder 474′ without frame 464′ and membrane 462′. FIGS. 7K-7L (and FIGS. 7A-7J) illustrate clamp 466′ in a first position (which is an example of a closed position) and FIGS. 7M-7O illustrate clamp 466′ in a second position (which is an example of an open position). Pivotable movement of clamp 466′ can advantageously facilitate securement of catheter hub 442 within catheter hub holder 474′ in a similar or identical manner as that described above with respect to clamp 466′ and catheter hub holder 474. Clamp 466′ can include one or more living hinges configured to allow clamp 466′ to pivot relative to frame 464′ (and membrane 462′). For example, clamp 466′ can include one or two or more living hinges. With reference to FIGS. 7R-7U, clamp 466′ can include a first living hinge 491′ having a first hinge portion 491a′, a second hinge portion 491b′, and a third hinge portion 491c′ that connects the first and second hinge portions 491a′, 491b′ to one another. Clamp 466′ can additionally include a second living hinge 493′ having a first hinge portion 493a′, a second hinge portion 493b′, and a third hinge portion 493c′ that connects the first and second hinge portions 493a′, 493b′ to one another.

Clamp 466′ can be pivotably secured to frame 464′. For example, in some implementations, clamp 466′ includes one or more posts 495a′, 495b′ configured to secure (for example, via a press-fit) within openings in a portion of frame 464. For example, posts 495a′, 495b′ can secure within holes 465a′ extending through a lip 465′ extending from (for example, inward from) frame body 470′ (see FIG. 7P). In some implementations, such as that shown in the figures, posts 495a′, 495b′ extend from second hinge portions 491b′, 493b′. Clamp 466′ can be secured to frame 464′ via securement of posts 495a′, 495b′ within openings 465a′ of frame 464′, and clamp 466′ can be configured to pivot between a plurality of positions, including but not limited to the open and closed positions shown in the figures.

In some implementations, clamp 466′ includes a biasing member configured to bias clamp 466′ towards a closed position (for example, a position where the latch portion 466b′ is closer to frame 464′/frame body 470′). For example, clamp 466′ can include biasing member 497′. Biasing member 497′ can be positioned proximate to living hinges 491′, 493′, for example, at an end of clamp 466′. Biasing member 497′ can act to urge clamp 466′ from a first position in which the first and second hinge portions 491a′, 491b′ are spaced from one another to a second position in which the first and second hinge portions 491a′, 491b′ are close together and/or contact one another. Additionally, the first and second hinge portions 493a′, 493b′ can be spaced from one another when clamp 466′ is in such first position and can be close together and/or contact one another when clamp 466′ is in such second position. Clamp 466′ can be secured to frame 464′ in an alternative manner than via securement of posts 495a′, 495b′ within openings 465a′. In some variants, clamp 466′ is permanently secured to frame 464′.

With continued reference to FIGS. 7R-7U, clamp 466′ can include cavity 466c′ and slots 466d′, which can be similar or identical to cavity 466 and slots 466 (respectively) of clamp 466.

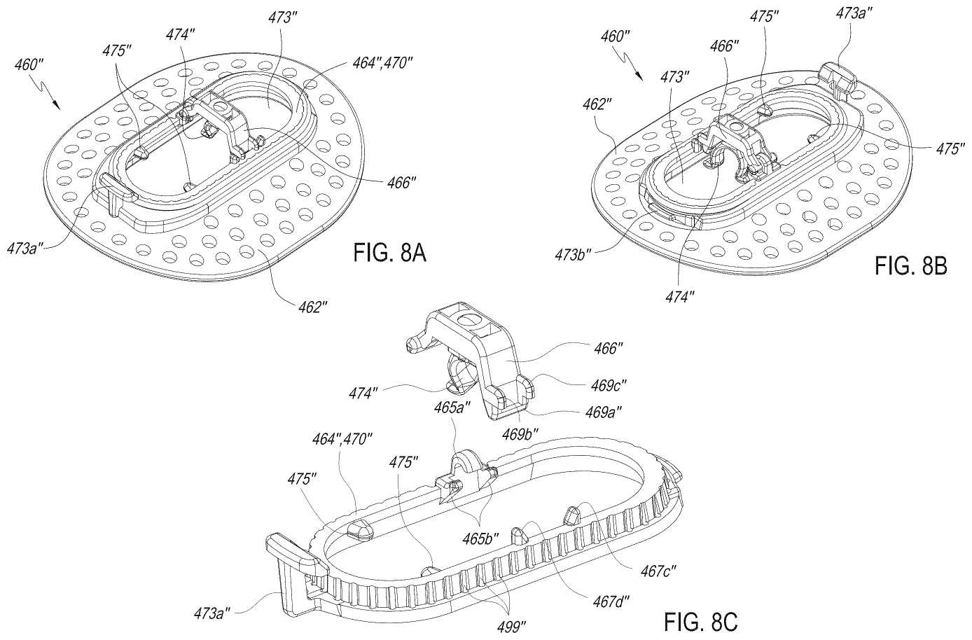

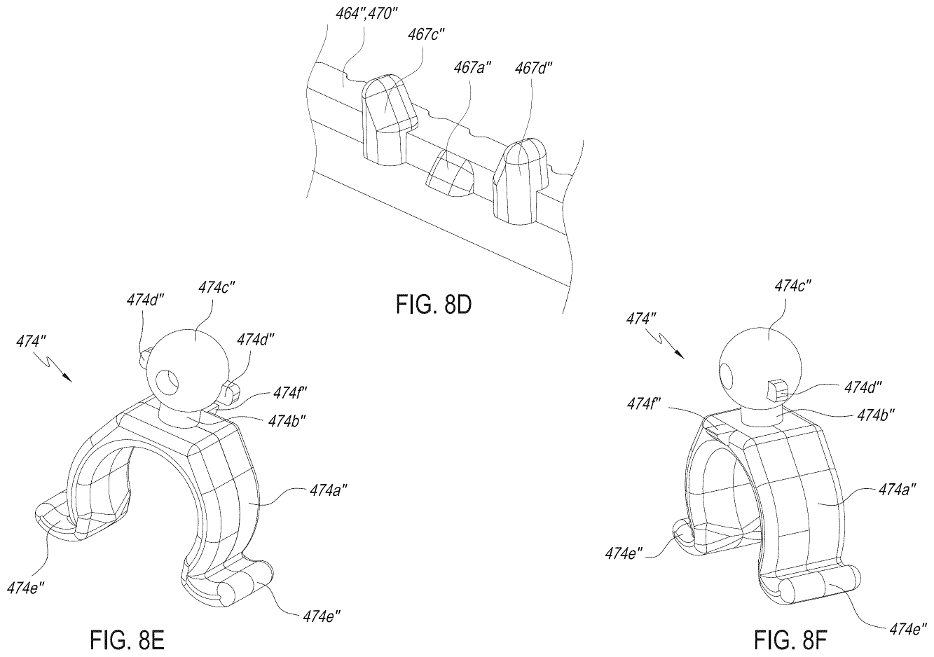

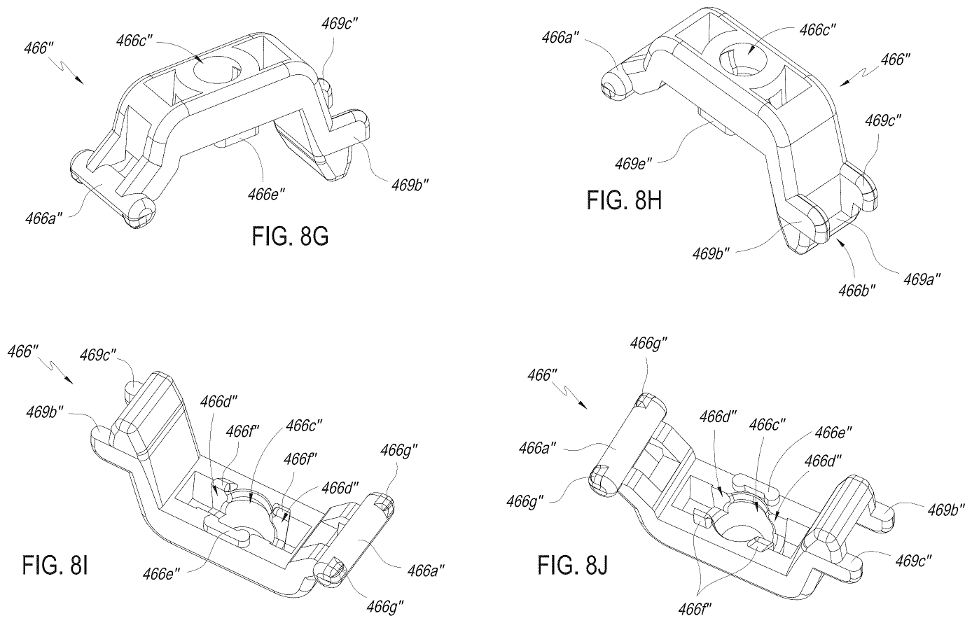

FIGS. 8A-8J illustrate another implementation of a hub 460″ that can be utilized with cover 420 and extension set 440 in a similar manner as that shown and/or described herein with respect to hub 460. Hub 460″ (which also may be referred to as a “dock” or “base”) can also be utilized with cover 520 and any of extension sets 540, 540′. Hub 460″ can be similar to hub 460 in some or many respects. For example, hub 460″ can include a membrane 462″ that can be identical to membrane 460. Hub 460″ can include a frame 464″ and frame body 470″ that can be similar to frame 464 and frame body 470 in many respects. Hub 460″ can include an opening 463″, a latch arm 473a″, a protrusion 473b″, and indicators 475″, each of which can be similar or identical to opening 463, latch arm 473a, protrusion 473b, and indicators 475 of hub 460 (respectively). Hub 460″ can include a clamp 466″ and a catheter hub holder 474″. FIGS. 8A-8B illustrate top perspective views of hub 460″. FIG. 8C illustrates a perspective view of frame 464″ separated from clamp 466″ and catheter hub holder 474″ (without showing membrane 462″). FIG. 8D illustrates an enlarged view of a portion of frame 464″. FIGS. 8E-8F illustrate perspective views of catheter hub holder 474″. FIGS. 8G-8J illustrate top and bottom perspective views of clamp 466″.

With reference to FIG. 8C, frame 464″ can include one or a plurality of grooves 499″ in frame body 470″ that can facilitate bonding of membrane 462″ and which can be similar or identical to grooves 499. As also shown, frame 464″ can include a hook 465a″ and/or one or both of protrusions 465b″ that can be configured to receive a pin 466a″ of clamp 466″ (see FIGS. 8G-8J) in a similar manner as that described above with respect to pin 466a, hook 465a, and protrusions 465b. Clamp 466″ can be configured to pivot relative to frame 464″ in a similar or identical manner as that described above with respect to clamp 466 and frame 464.

With reference to FIGS. 8G-8J, clamp 466″ can include a pin 466a″ (for example, at a first end of clamp 466″) and a latch portion 466b″ (for example, at a second end of clamp 466″). Latch portion 466b″ can be configured to engage a portion of frame 464″ in a similar manner as that described above with respect to latch portion 466b and frame 464. Latch portion 466b″ can include a ledge 469a″ that can be configured to interact with (for example, secure to) protrusion 467a″ extending from frame body 470″ (see FIG. 8D) in a similar manner as that described above with respect to ledge 469a and protrusion 467a. Protrusion 467a″ can be tapered and/or can inhibit disconnection of latch portion 466b″ from frame 464″ in a similar or identical manner as that described above with respect to latch portion 466b and frame 464. Latch portion 466b″ can include one or both of nubs 469b″, 469c″ that can be configured to interact with and/or be positioned between protrusions 467c″, 467d″ extending from frame body 470″. Protrusions 467c″, 467d″ (which may be referred to as “guide protrusions” or “alignment protrusions”) can be spaced from one another and can act as a guide to aid in placement of latch portion 466b″. Protrusions 467c″, 467d″ can have a tapered end (see FIG. 8D) that can contact nubs 469b″, 469c″ when latch portion 466b″ is moved to a closed position (such as that shown in FIGS. 8A-8B) and can aid in positioning and/or alignment of protrusion 467a″ with ledge 469a″. With reference to FIGS. 8A and 8C, nubs 469b″, 469c″ can be configured to rest atop frame body 470″ when clamp 466″ is in a closed position, such as that illustrated in FIGS. 8A-8B. With reference to FIG. 4OO and FIG. 8H, latch portion 466b″ of clamp 466″ can have a smaller width than latch portion 466b of clamp 466. Additionally, with reference to FIG. 4KK and FIG. 8D, a distance between protrusions 467c″ and 467d″ can be smaller than a distance between protrusions 467c and 467d, each of which are sized to accommodate latch portions 466b″ and 466b (respectively). A shorter width of latch portion 466b″ and distance between protrusions 467c″ and 467d″ can provide more space for, for example, male connector 443 when connected (alone or along with extension set 440).

FIGS. 8E-8F illustrate perspective views of catheter hub holder 474″. Catheter hub holder 474″ can be similar or identical to catheter hub holder 474 in some or many respects. Catheter hub holder 474″ can include a body 474a″, a ball 474c″, a stem 474b″, ear(s) 474d″, and/or arms 474e″ each of which can be similar or identical (respectively) to body 474a, ball 474c, stem 474b, and arms 474e described above.

With continued reference to FIGS. 8G-8J, clamp 466″ can include a cavity 466c″ that can be configured to receive ball 474a″ of catheter hub holder 474″. Cavity 466c″ can be similar or identical to cavity 466 of clamp 466. Clamp 466″ can also include one or both of slots 466d″ that can be configured to receive ears 474d″ of catheter hub holder 474″ in a similar or identical manner as that described above with respect to slots 466d and ears 474d. Ears 474d″ can be positioned within slots 466d″ and can be configured to limit rotation of ball 474c″ in a similar or identical manner as that described above with respect to ears 474d and slots 466d. In some variants, catheter hub holder 474″ does not include ears 474d″. As shown, clamp 466″ can include bumps 466g″ extending from pin 466a″ that can be similar or identical to bumps 466g.

In addition or as an alternative to ear(s) 474d″ and slots 466d″, catheter hub holder 474″ and clamp 466″ can include other structure that can limit rotation of catheter hub holder 474″ relative to clamp 466″. Limiting rotation of catheter hub holder 474″ relative to clamp 466″ can in turn limit rotation of catheter hub 442 relative to clamp 466″, frame 464″, and/or membrane 462″, when catheter hub 442 is connected to catheter hub holder 474″. With reference to FIGS. 8E-8F and FIGS. 8I-8J, catheter hub holder 474″ can include a protrusion 474f″ that can be configured to engage with a stop 466e″ of clamp 466″ to limit rotation of catheter hub holder 474″ relative to clamp 466″. In addition or as an alternative to stop 466e″, clamp 466″ can include protrusions 466f″. In some implementations, protrusions 466f″ have a tapered shape. Stop 466e″ and protrusions 466f″ can limit movement of catheter hub holder 474″ relative to clamp 466″ in a similar manner as that described above with respect to protrusions 466e, 466f. In some variants, catheter hub holder 474″ does not include protrusion 474f″. In some variants, clamp 466″ does not include stop 466e″ and/or does not include protrusions 466f″.

### Additional Considerations

Although this disclosure has been described in the context of certain examples, it will be understood by those skilled in the art that the present disclosure extends beyond the specifically disclosed examples to other alternative examples and/or uses of the disclosure and obvious modifications and equivalents thereof. In addition, while a number of variations of the disclosure have been shown and described in detail, other modifications, which are within the scope of this disclosure, will be readily apparent to those of skill in the art based upon this disclosure. It is also contemplated that various combinations or subcombinations of the specific features and aspects of the examples may be made and still fall within the scope of the disclosure. Accordingly, it should be understood that various features and aspects of the disclosure can be combined with or substituted for one another in order to form varying modes of the disclosed examples.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments.

Certain terminology may be used in the following description for the purpose of reference only, and thus are not intended to be limiting. For example, terms such as “above” and “below” refer to directions in the drawings to which reference is made. Terms such as “proximal,” “distal,” “front,” “back,” “rear,” and “side” describe the orientation and/or location of portions of the components or elements within a consistent but arbitrary frame of reference which is made clear by reference to the text and the associated drawings describing the components or elements under discussion. Such terminology can include the words specifically mentioned above, derivatives thereof, and words of similar import.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

While the above detailed description has shown, described, and pointed out novel features, it can be understood that various omissions, substitutions, and changes in the form and details of the devices or systems illustrated can be made without departing from the spirit of the disclosure. As can be recognized, certain portions of the description herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

1. A device configured for use at a catheter insertion site of a subject, the device comprising:
a base comprising an opening, the base configured to be secured to skin of the subject such that said opening is positioned over the catheter insertion site; and
a catheter hub holder configured to secure to a catheter hub connected to a catheter that is configured for insertion at the catheter insertion site;
wherein the catheter hub holder is movably coupled to the base to allow for relative movement between the base and the catheter hub and catheter when said catheter is inserted at the catheter insertion site.

2. The device of claim 1, wherein the catheter hub holder is movably coupled to the base via a gimbal arrangement.

3. The device of claim 1, wherein the catheter hub holder is rotatably coupled to the base and configured to rotate at least partially about three, mutually orthogonal axes.

4. The device of claim 3, wherein the catheter hub holder comprises a ball and the base comprises a cavity configured to receive said ball, and wherein said ball is rotatable within said cavity.

5. The device of claim 4, wherein the catheter hub holder further comprises:
a body configured to surround and secure to a portion of said catheter hub; and
a stem connected to and extending between the body and said ball such that the ball is spaced from the body.

6. (canceled)

7. (canceled)

8. (canceled)

9. (canceled)

10. (canceled)

11. (canceled)

12. (canceled)

13. (canceled)

14. (canceled)

15. (canceled)

16. The device of claim 1, wherein:
the base comprises:
a membrane configured to secure to the subject's skin, the membrane comprising said opening of the base; and
a frame connected to the membrane along at least a portion of a perimeter of said opening; and
the catheter hub holder is movably coupled to said frame.

17. The device of claim 16, wherein the base further comprises a brace connected to the frame and extending across said opening, and wherein the catheter hub holder is movably coupled to the frame via the brace.

18. The device of claim 17, wherein the frame comprises a frame body having an at least partially annular shape, and wherein the brace comprises a first end connected to a first side of the frame body and a second end connected to a second side of the frame body, said second side of the frame body arranged opposite the first side of the frame body.

19. The device of claim 17, further comprising a cover configured to removably secure to the frame and enclose the brace, the catheter hub holder, and the catheter insertion site.

20. The device of claim 17, wherein the catheter hub holder comprises a ball and the brace comprises a cavity configured to receive said ball, and wherein said ball is rotatable within said cavity to permit rotation of the catheter hub holder relative to the brace.

21. The device of claim 20, wherein:
the brace comprises at least one slot;
the catheter hub holder further comprises at least one ear extending outward from the ball and positioned within the at least one slot of the brace; and
contact between (i) portions of the brace forming said at least one slot and (ii) the at least one ear limits an amount of rotation of the catheter hub holder relative to the brace about at least one of three, mutually orthogonal axes.

22. The device of claim 16, wherein the frame comprises a more rigid material than the membrane.

23. A device configured for use at a catheter insertion site of a subject, the device comprising:
a base configured to be secured to skin of the subject around the catheter insertion site, the base comprising:
a membrane comprising an opening configured to be positioned over the catheter insertion site when the base is secured to the subject's skin;
a frame connected to the membrane along at least a portion of a perimeter of the opening;
a brace extending over the opening of the membrane and connected to the frame; and
a catheter hub holder configured to secure to a catheter hub connected to a catheter that is configured for insertion at the catheter insertion site, the catheter hub holder movably coupled to the brace and arranged over the opening of the membrane; and
a cover configured to removably secure to the base and at least partially enclose the catheter insertion site.

24. The device of claim 23, wherein the frame comprises a frame body having an at least partially annular shape, and wherein the brace comprises a first end connected to a first side of the frame body and a second end connected to a second side of the frame body, said second side of the frame body arranged opposite the first side of the frame body.

25. The device of claim 23, wherein the cover is configured to removably secure to the frame via one or more latch mechanisms.

26. The device of claim 23, wherein the catheter hub holder comprises a ball and the brace comprises a cavity configured to receive said ball, and wherein said ball is rotatable within said cavity to permit rotation of the catheter hub holder relative to the brace.

27. The device of claim 26, wherein:
the brace comprises at least one slot;
the catheter hub holder further comprises at least one ear extending outward from the ball and positioned within the at least one slot of the brace; and
contact between (i) portions of the brace forming said at least one slot and (ii) the at least one ear limits an amount of rotation of the catheter hub holder relative to the brace about at least one of three, mutually orthogonal axes.

28. The device of claim 26, wherein the catheter hub holder further comprises:
an at least partially C-shaped body configured to surround and secure to a portion of said catheter hub; and
a stem connected to and extending between the body and said ball such that the ball is spaced from the body.

29. The device of claim 23, wherein the brace is to removably connected to the frame.

30. The device of claim 23, wherein the frame comprises a more rigid material than the membrane.
